(19) United States
(12) Patent Application Publication
(10) Pub. No.: US 2012/0251614 A1
(43) Pub. Date: Oct. 4, 2012

(54) PROCESSED FEEDS, FOODS AND BIOFUELS AND METHODS OF MAKING AND USING THEM

(75) Inventor: Gary ROHWER, Parma, ID (US)

(73) Assignee: Parma Laboratories, Inc, Goodyear, AZ (US)

(21) Appl. No.: 13/286,822

(22) Filed: Nov. 1, 2011

Related U.S. Application Data
(63) Continuation of application No. PCT/US2010/032434, filed on Apr. 26, 2010.
(60) Provisional application No. 61/174,917, filed on May 1, 2009.

(51) Int. Cl. A61K 9/127; A61K 38/00; C07K 14/435; A23J 3/00; C07K 1/36; C07K 14/415

(52) U.S. Cl. 424/450; 530/422; 530/370; 426/424; 514/1.1

(57) ABSTRACT

In one aspect, the invention provides methods for manufacturing, processing and recovering organic matter from a biomass, e.g., a biofluid or biosemisolid comprising a protein and a lipid, for example, from a dairy-based material. In one aspect, the invention provides methods for encapsulating oils with proteins for, e.g., making foods, food supplements or additives and feeds or feed supplements or additives. Compositions made by processes of this invention can be used for foods, food supplements or additives, feeds or feed supplements or additives, and/or biofuels.

### TECHNICAL FIELD

This invention relates to foods, feeds and fuel processing and manufacture. In one aspect, the invention provides methods for manufacturing, processing and recovering organic matter from a biomass, e.g., a biofluid or biosemisolid comprising a protein and a lipid, for example, from a dairy-based material. In one aspect, the invention provides methods for encapsulating oils with proteins for, e.g., making foods, food supplements or additives and feeds or feed supplements or additives. Compositions made by processes of this invention can be used for foods, food supplements or additives, feeds or feed supplements or additives, and/or biofuels. In one-aspect, the invention provides methods that can be an effective recycling process to destroy infectious agents, e.g., prions, while at the same time provide methods for recovering valuable animal proteins for e.g., animal feeds.

### BACKGROUND

Current methods of producing protein encapsulated lipids have various problems in production. These include lack of complete encapsulation of the lipid, use of chemicals, such a formaldehyde, that have production and regulatory issues, and high moisture processing conditions-because of processing requirements or high moisture in source materials.

### SUMMARY

The invention provides methods for processing and recovering organic matter from a biofluid, biosemisolid or biosolid (biomass) comprising a protein and a lipid, comprising:

(a) providing a first liquid and/or semisolid or paste comprising an organic matter (a biomass) in a liquid, a semisolid or paste, a colloidal and/or a solid or a particulate form;

(b) adding to the first liquid and/or semisolid or paste of step (a) a second liquid comprising a base to raise the pH (make the liquid and/or semisolid or paste more alkaline) of the first and second liquid mixture to:

(i) between about pH 8 and about pH 13, or between about pH 11 and about pH 12, or at least about pH 8, about pH 9, about pH 10, about pH 11, about pH 12 or about pH 13,

(ii) a point at which the protein flocculates or coagulates, or the protein and lipid form an emulsion, or

(iii) a point at which the protein and lipid form a lipid-encapsulated structure (a liposome or vesicle comprising the lipid encapsulating the protein) or a protein-encapsulated structure (a liposome or vesicle comprising the protein encapsulating the lipid),

and raising the temperature of the first liquid and/or semisolid or paste to a point at or above the melting point of the lipid, wherein the temperature is raised either before, during and/or after adding the second liquid to the first liquid;

(c) mixing, and shearing or sonicating the liquid of step (b), and at the same time or any time during the process of the mixing, or shearing or sonicating, adding a liquid comprising an acid to:
(i) lower the pH (make the liquid more acid) to between about pH 3 and pH 6, or about pH 3 or less, about pH 4, about pH 5, or about pH 6, thereby generating a precipitate or flocculant comprising substantially most of the protein, lipid or lipid-encapsulated structures or protein-encapsulated structures out of the liquid, or
(ii) lower the pH (make the liquid more acid) to the point where substantially most of the protein, lipid, or lipid-encapsulated structures or protein-encapsulated structures precipitate or flocculate out of the liquid; and

(d) separating or removing some, all or substantially most of the liquid from the precipitate or flocculate or separating or removing some, all or substantially most of the precipitate or flocculate from the liquid.

In alternative embodiments of the methods:

(a) the first liquid and/or semisolid or paste comprising an organic matter (a biomass) comprises a mixture of carbohydrates, proteins and lipids; or

(b) the colloidal form comprises a colloidal aerosol, a colloidal emulsion, a colloidal foam, a colloidal dispersion or a hydrosol.

In alternative embodiments of the methods, the first liquid and/or semisolid or paste comprising an organic matter (a biomass) comprises an animal-based or an animal-derived organic matter or a plant-based or a plant-derived organic matter or a microorganism-based or a microorganism-derived organic matter or an algae-based or an algae-derived organic matter; or any combination thereof.

In alternative embodiments of the methods, the animal-based or animal-derived liquid organic matter comprises one or more dairy or meat products dairy-derived or meat-derived products, or one or more fish or fish-derived products.

In alternative embodiments of the methods, the one or more dairy products comprises milk, cheese, cream cheese, cottage cheese, yogurt or sour cream, or any combination thereof; or, the one or more dairy products comprises LIPITEIN®.

In alternative embodiments of the methods, the protein-encapsulated structure (liposome or vesicle comprising-protein encapsulating lipid) comprises:

(a) a plant-derived or an animal-derived protein encapsulating an animal-, plant- or fish-derived oil;

(b) a soy-derived or dairy-derived protein encapsulating an animal-, plant- or fish-derived oil;

(c) the method of (a) or (b), wherein the dairy-derived protein comprises a whey (milk plasma) or equivalent; or the plant-derived oil comprises a flax oil or linseed oil (flax seed oil).

In alternative embodiments of the methods, the plant-based or plant-derived first liquid and/or semisolid or paste organic matter (biomass), or the algae-based or algae-derived liquid and/or semisolid or paste organic matter (biomass), comprises:

(a) a rice, flaxseed (linseed), rapeseed (canola), sunflower, cottonseed, peanut, wheat, soy, barley, sorghum, oats, potato or corn plant, product or extract, or any combination thereof;

(b) a plant, product or extract from a species from a genera Anacardium, Arachis, Asparagus, Atropa, Avena, Brassica, Citrus, Citrullus, Capsicum, Carthamus, Cocos, Coffea, Cucumnis, Cucurbita, Daucus, Elaeis, Fragaria, Glycine, Gossypinum, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Phormium, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, or Zea, or any combination thereof;

(c) a plant, product or extract from a cotton, a silk cotton tree (Kapok, Ceiba pentandra), desert willow, a creosote bush, a winterfat, a balsa, a ramic, a kenaf, a hemp, a roselle, a jute, a sisal abaca, a flax, a member of the Linaceae family, a Linum usitatissimum, a Phormium tenax or a Phormium cookianum or any combination thereof;

(d) a composition, product or extract comprising or derived from a macroalgae, a seaweed; a red seaweed; a specie of the genus Ulva, Chlorophyta, Charophyta, Rhodophyta, Phaeophyceae, Porphyra, Gracilaria, Grateloupia, Kappaphycus or Ceramium; a green seaweed; a brown seaweed or brown algae; a kelp; a specie of the genus Laminaria; or, a Laminaria japonica;

(e) a plant, composition, product or extract comprising or derived from a halophyte, a plant or specie of the genus Salicornia or Spartina, or a Spartina alterniflora (smooth cordgrass or saltmarsh cordgrass) or a Spartina patens (saltmeadow cordgrass); or

(f) a composition, product or extract comprising or derived from a microalgae (also called phytoplankton, microphytes, planktonic algae).

In alternative embodiments of the methods, the plant-based or plant-derived first liquid and/or semisolid or paste organic matter (biomass) comprises a plant, food, fish, fowl or poultry, or animal waste stream; or a plant, food, fowl or poultry, fish or animal processing waste stream, or a human, fowl or poultry, fish or animal excrement (sewage) processing plant waste stream, or any combination thereof.

In alternative embodiments of the methods, the first liquid and/or semisolid or paste organic matter (biomass) comprising the animal-derived, plant-based or plant-derived liquid organic matter is manufactured by a process comprising: (a) raising the pH to at least pH 10.2; (b) raising the pH to between about pH 10.2 and pH 11.5; or, (c) the method of (a) or (b), and wherein at least one step of the process comprises raising: the temperature to between about 140° C. and 1-80° C.

In alternative embodiments of the methods, in step (b) the pH is raised (make the liquid more alkaline) to about pH 11.0, pH 11.1, pH 11.2, pH 11.3, pH 11.4, pH 11.5 or pH 11.6.

In alternative embodiments of the methods, the base added in step (b) comprises a calcium hydroxide, or a Ca(OH₂) (or slaked lime or hydrated lime), a sodium hydroxide (lye or caustic soda), a potassium hydroxide, a calcium oxide, a magnesium hydroxide, or an alkaline solution (an alkali), or any combination thereof.

In alternative aspects, the methods further comprise, e.g. after step (b), adding to the raised pH liquid a third liquid comprising an organic matter and a buffer in an amount sufficient to lower the pH (make the liquid more acid) in any amount that lowers the pH (makes the liquid more acidic) to an approximately neutral pH (or about pH 7). The third liquid can comprise a milk, or a milk-based or milk-derived liquid.

In alternative embodiments of the methods, the mixing or shearing of step (c) comprises a high shear mixing or a high-shear homogenization in a mixing or a shearing device. The mixing or shearing can comprise:

(a) pumping the liquid of step (b) to the mixing or shearing device with a line pressure above about 25 psi., or in the range of 60 to 150 psi;

(b) using a HYDRASHEAR™ shearing device;

(c) using an in-line injector immediately following the mixing or shearing to add the liquid comprising an acid; or

(d) the method of (c), wherein sufficient acid is added to lower the pH (acidify) to a pH of protein-lipid encapsulation, or a lower (more acid) pH.

In alternative embodiments of the methods, the liquid comprising an acid added in the mixing of step (c) comprises a phosphoric acid, a nitric acid, a boric acid, a hydrochloric acid, a sulfuric acid, a lactic acid, an acetic acid, a propionic acid (propanoic acid), a formic acid, a citric acid, an oxalic acid, or an organic acid, or any combination thereof.

In alternative embodiments of the methods, the pH of the liquid in step (c) is lowered (made more acidic) to about pH 2.8, pH 2.9, pH 3.0, pH 3.1, pH 3.2, pH 3.3, pH 3.4, pH 3.5, pH 3.6, pH 3.7, pH 3.8; pH 3.9, pH 4.0, pH 4.1, pH 4.2, pH 4.3, pH 4.4, pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7 or pH 5.8.

In alternative embodiments of the methods, separating or removing the liquid from the precipitate or flocculate in step (d) comprises centrifugation, filtering, chromatography, or any combination thereof.

In alternative aspects the methods further comprise a step (e) comprising treating the liquid of step 1(d) from which the precipitate or flocculate has been separated/removed by an additional protein and/or lipid separation procedure, wherein the additional protein and/or lipid separation procedure step removes or separates solids, protein and/or lipid not separated or removed by the step 1(d) separation. The additional protein and/or lipid separation procedure can comprise precipitation, centrifugation, filtering, chromatography, electrophoresis, or a combination thereof. The filtering can comprise filtering with a screen or vacuum screen, a membrane, or filtering by ultrafiltration or nanofiltration; e.g., the filtering with a membrane can comprise filtering with a polyvinylidene fluoride (PVDF) membrane or a microporous membrane, or any combination thereof. The PVDF can comprise a UF-PVDF-30K membrane. In alternative embodiments of the methods, the microporous membrane comprises a pore size ranging between about 0.1 to 10 micrometers (μm).

The ultrafiltration can comprise filtering with a selectively permeable membrane, a partially-permeable membrane or a differentially-permeable membrane, or any combination thereof. The ultrafiltration can comprise filtering with a cellulose ester membrane (CEM), a charge mosaic membrane (CMM), a bipolar membrane (BPM), an anion exchange membrane (AEM), an alkali anion exchange membrane (AAEM) or a proton exchange membrane (PEM), or any combination thereof.

In alternative embodiments of the methods, the precipitation comprises precipitating protein not separated or removed by the step 1(d) separation with an ammonium sulfate ((NH₄)₂SO₄,) solution, or equivalent.

In alternative, embodiments of the methods, the centrifugation comprises a density gradient centrifugation, an equilibrium centrifugation or a non-equilibrium centrifugation, or any combination thereof. The chromatography can comprise a size exclusion chromatography, an ion exchange chromatography, an affinity chromatography, a high performance liquid chromatography (HPLC) or a high pressure liquid chromatography, or any combination thereof. In alternative embodiments of the methods, the electrophoresis comprises a denaturing-condition electrophoresis or a non-denaturing-condition electrophoresis, or any combination thereof.

In alternative aspects, the methods further comprise a step (f) comprising drying proteins, lipids and/or solids not initially separated/removed by the step (d) separation, but separated/removed in step (e). In alternative aspects, the methods further comprise a step (e) comprising drying proteins, lipids and/or solids not separated or removed when the precipitate or flocculate was separated or removed from the liquid. The drying can comprise lyophilization (freeze-drying or cryodesiccation), vacuum drying, vacuum kiln drying, evaporation, application of heated air (convective or direct drying), indirect or contact drying (heating through a hot wall), drum drying, supercritical drying (superheated, steam drying), spray drying, natural air (drying by treating with unheated forced air), or any combination thereof.

In alternative aspects, the methods further comprise a step (f) comprising concentrating the protein, precipitate or flocculate by lyophilization (freeze-drying or cryodesiccation), ultrafiltration, or any combination thereof.

In alternative aspects, the methods further comprise a step (e) comprising concentrating the protein, precipitate or flocculate by lyophilization (freeze-drying or cryodesiccation) or ultrafiltration, or any combination thereof.

The invention provides methods for processing and recovering organic matter from a dairy-based, soy-based and/or a fruit-based liquid, comprising:

(a) providing a dairy-based, soy-based and/or a fruit-based first liquid, colloid and/or semisolid or paste, wherein the dairy-based and/or a fruit-based first liquid, colloid and/or semisolid or paste comprise a protein and a lipid;

(b) adding to the dairy-based and/or a fruit-based first liquid, colloid and/or Semisolid or paste of step (a) a second liquid comprising a base comprising a sodium hydroxide, a potassium hydroxide or a calcium hydroxide or any combination thereof to raise the pH (make the liquid and/or semisolid or paste more alkaline) to:
(i) between about pH 9.5 and about pH 10.5,
(ii) a point at which substantially most of the protein flocculates or coagulates, or the protein and lipid form an emulsion, or
(iii) a point at which the protein and lipid form a lipid-encapsulated structure (a liposome or vesicle comprising the protein) or a protein-encapsulated structure,
and raising the temperature to: (i) at or above the melting point of milk fat, or to between about 120° F. to about 160° F., either before, during and/or after adding the base-comprising second liquid to the first liquid; and

(c) mixing and shearing the liquid of step (b), and at the same time or any time during the process of the mixing and shearing or sonicating, adding a liquid comprising an acid comprising a phosphoric acid to:
(i) lower the pH (make the liquid more acid) to: or between about pH to 3.0 to about 6.5, or between about pH 6.1 to about pH 6.3, or between about pH to 4.5 to about pH 3.3, thereby generating a precipitate or flocculant comprising substantially most of the protein, lipid or lipid-encapsulated structures protein-encapsulated structures out of the liquid, or
(ii) lower the pH (make the liquid more acid) to the point where substantially most of the protein, lipid, or lipid-encapsulated structures or protein-encapsulated structures precipitate or flocculate out of the liquid;

(d) separating or removing the liquid from the precipitate or flocculate, or separating or removing the precipitate or flocculate from the liquid.

In alliterative aspects, the methods further comprise screening the dairy-based and/or a fruit-based first liquid, colloid and/or semisolid or paste, and/or the base-adjusted liquid of step (b), and/or the acid-adjusted liquid of step (c), to remove solid contaminant particles. The screening can comprise screening out (removing) all or substantially most of the particles (e.g., foreign materials such as plastic, container plastic and/or ungular (hoof, claw or talon) parts or other foreign materials) from the liquid, colloid, and/or semisolid or paste, and/or the base-adjusted liquid. Bar, vibratory, basket, horizontal belt power augur and other screens of appropriate sizing as well as incline augurs, settling basins can be used to remove foreign materials.

The precipitate or flocculate can be separated or removed from the liquid by: filtering under through an at least 200 mesh (or finer) screen; filtering under-vacuum through a 200 mesh (or finer) stainless screen; or, by centrifugation.

The precipitate or flocculate can be separated or removed from the liquid by: filtering under through stainless screens between about 60 and 400 mesh; filtering under vacuum through stainless screens between about 60 and 400 mesh under vacuum; and/or, by centrifugation. The precipitate or flocculate can be separated or removed from the liquid by: filtering or filtering under vacuum using other appropriate filter and/or membrane materials; and/or by centrifugation. In alternative embodiments, the precipitate or flocculate is separated or removed from the liquid by using: a centrifuge, or a commercial liquid/solids ejector centrifuge; or by using a decanter centrifuge, a decanter, a di-decanter or a tri-decanter, or a liquid/liquid separation, and/or by using a centrifuge. The precipitate or flocculate can be separated or removed from the liquid by centrifuging at a temperature of between about 5° C. to about 55° C. at a pH or between about 3.3 and 5.8 using a commercial liquid/solids ejector centrifuge.

In alternative aspects, the methods further comprise lowering the pH of the final liquid product of step (d) to: between about pH to 5.0 to about pH 3.0 or less, or between about pH to 4.5 to about pH 3.3. In alternative aspects, the methods further comprise filtering the final liquid product of step (d). The filtering can comprises a membrane filtration, e.g., the membrane filtration can comprise filtering across a UF-PVDF-30K membrane.

In alternative aspects, the methods further comprise cooling the final liquid product of step (d); or further comprising cooling the final liquid product of step (d) to between about 35° F. to about 42° F.

In alternative aspects, the methods further comprise drying the removed precipitate or flocculate to a semi-solid, paste or solid form, or drying the removed precipitate or flocculate to a semi-solid, paste or solid form on a commercial tray dryer.

The invention provides methods for processing and recovering organic matter from a dairy-based, soy-based and/or a fruit-based liquid, comprising:

(a) providing a dairy-based, soy-based and/or a fruit-based first liquid, colloid and/or semisolid or paste, wherein the first liquid, colloid and/or semisolid or paste comprises a protein;

(b) adding to the dairy-based, soy-based and/or a fruit-based first liquid, colloid and/or semisolid or paste of step (a) a second liquid comprising a base comprising a sodium hydroxide, a potassium hydroxide or a calcium hydroxide or any combination thereof to raise the pH (make the liquid and/or semisolid or paste more alkaline) to:
(i) between about pH 9.5 and about pH 10.5, or between about pH 10.5 and about pH 11.5, or
(ii) a point at which substantially most of the protein flocculates or coagulates;

and raising the temperature to: (i) at or above the melting point of milk fat, or to between about 120° F. to about 160° F., or between about 90° F. to about 130° F., either before, during and/or after adding the base-comprising second liquid to the first liquid;

(c) adding to and mixing into the raised temperature liquid of step (c) a preheated (prewarmed) liquefied fat or lipid, wherein the fat or lipid is preheated (prewarmed) to a temperature: (i) at or above the melting point of milk fat, or to between about 120° F. to about 160° F.;

(d) mixing and shearing the liquid of step (c), and at the same time or any time during the process of the mixing and shearing or sonicating, adding a liquid comprising an acid comprising a phosphoric acid to:
(i) lower the pH (make the liquid more acid) to: or between about pH to 3.0 to about 4.0, or between about pH 3.6 to about pH 4.2, or about pH 3.6, pH 3.7, pH 3.8, pH 3.9 or pH 4.0, or about pH 3.0 or less, thereby generating a precipitate or flocculant, or
(ii) lower the pH (make the liquid more acid) to the point where substantially most of the protein, fat or lipid, or fat/lipid-encapsulated structures or protein-encapsulated structures precipitate or flocculate out of the liquid; and

(e) separating or removing the liquid from the precipitate or flocculate, or separating or removing the precipitate or flocculate from the liquid.

In alternative embodiments, the preheated (prewarmed) liquefied fat or lipid comprises an animal tallow, a suet; a lard, a rendered or unrendered fat, yellow grease (including frying oils from deep fryers and restaurants grease traps, or lower-quality grades of tallow from rendering plants), a vegetable oil, a waste vegetable oil (WVO), a straight vegetable oil (SVO) or a pure plant oil (PPO); or a brown grease or any grease or fat contaminated and considered unsuitable for food re-use; or a biodiesel oil; or an algae-derived oil or lipid.

In alternative embodiments, the mixing and shearing step comprises pumping the warmed liquid comprising the fat or lipid past (through) one or more shearing devices inline; and phosphoric acid is injected under pH control.

In alternative embodiments, the shearing device comprises a HYDRASHEAR™ device or equivalent device.

In alternative embodiments, the precipitate or flocculate is separated or removed from the liquid by using a centrifuge, or a commercial liquid/solids ejector centrifuge.

In alternative embodiments, the methods further comprise drying the removed precipitate or flocculate to a semi-solid, paste or solid form, or drying the removed precipitate or flocculate to a semi-solid, paste or solid form on a dryer or a commercial tray dryer.

In alternative embodiments, the methods further comprise screening the dairy-based, soy-based and/or a fruit-based first liquid, colloid and/or semisolid or paste, and/or the base-adjusted liquid of step (b), the raised temperature liquid of step (b), and/or the acid-adjusted liquid of step (d), to remove solid contaminant particles. The screening can comprise screening out of particles of plastic, container plastic and/or ungular (hoof, claw or talon) parts.

The invention provides methods for processing and recovering organic matter from a dairy-based composition, comprising:

(a) providing a dairy-based solid, colloid and/or semisolid or paste composition, wherein optionally the dairy-based solid, colloid and/or semisolid or paste composition is in its wholesale or retail packaging containers;

(b) crushing or mashing the dairy-based solid, colloid and/or semisolid or paste composition, and optionally screening out crushed pieces of the packaging containers;

(c) spraying over the crushed or mashed solid, colloid and/or semisolid or paste composition of step (b) a second liquid comprising the heated liquid of step (b), wherein the temperature of the sprayed heated liquid of step (b) is: (i) at or above the melting point of milk fat, or to between about 120° F. to about 160° F., or between about 90° F. to about 130° F., thereby producing a combined liquid products and solid products stream at between about pH 11.0 to 11.5, and at a temperature of between about 90° F. to about 130° F.; and

(d) collecting the combined liquid products and solid products stream in a holding container or holding tank.

In alternative embodiments, the dairy-based solid, colloid and/or semisolid or paste composition comprises about one third yogurt, about one third sour cream, and about one third cottage cheese.

In alternative embodiments, methods further comprise measuring the pH of the combined products of step (d), and if the pH is below pH 9.5, then sodium hydroxide or potassium hydroxide or calcium hydroxide or a combination thereof is added to obtain a pH of between 9.5 and 10.5, or between about pH 10.0 and pH 11.0.

In alternative embodiments, methods further comprise heating combined the combined products of step (d) above the melting point of the milk fat or to between about 120° F. to 160° F., and thoroughly mixing, and screening to remove small particles of packaging materials.

In alternative embodiments, methods further comprise passing or pumping the liquid through a shearing mechanism to perform a shearing step. The shearing step can comprise pumping the warmed liquid past (through) one or more shearing devices inline; and phosphoric acid is injected under pH control. The shearing device can comprise a HYDRASHEAR™ device or equivalent device.

In alternative embodiments, the pH of the liquid is adjusted to between about 3.0 and about pH 5.6, or to about pH 3.0, pH 3.1, pH 3.2, pH 3.3, pH 3.4, pH 3.5, pH 3.6, pH 3.7, pH 3.8, pH 3.9, pH 4.0, pH 4.1, pH 4.2, pH 4.3, pH 4.4, pH 4.5, pH 4.6, pH 4.7, pH 4.8, pH 4.9, pH 5.0, pH 5.1, pH 5.2, pH 5.3, pH 5.4, pH 5.5 or pH 5.6 or higher, and in one embodiment is centrifuged, e.g., using a commercial centrifuge, thereby recovering solids. The solids can be dried, or the solids can be dried on a commercial tray dryer.

The invention provides methods for producing a biofuel comprising providing an organic matter or a biomass and treating the organic matter or biomass using a process comprising a method of the invention. The biofuels made by these methods can comprise a bioalcohol or a biodiesel.

The invention provides methods for producing a food or feed, or a food or feed additive or supplement, a nutritional or dietary supplement, comprising providing an organic matter or a biomass and treating the organic matter or biomass using a process comprising a method of the invention. The food or feed, or food or feed additive or supplement, or nutritional or dietary supplement, can be formulated or made for a human, a domestic or zoo animal, an ungulate, or a horse, a goat, a sheep, a cow or a calf, or for cattle or bison.

The invention provides food or foods, feeds, food or feed additives or supplements, or nutritional or dietary supplements, comprising an organic matter (a biomass) made or processed by a method of the invention. In alternative aspects of the food or a feed, or a food or feed additive or supplement, or nutritional or dietary supplement, the processed organic matter (biomass) comprises an animal-based or an animal-derived organic matter or a plant-based or a plant-derived organic matter or a microorganism-based or a microorganism-derived organic matter or an algae-based or an algae-derived organic matter, or any combination thereof. The processed organic matter (biomass) can comprise an animal-based or animal-derived liquid organic matter, or one or more dairy or meat products dairy-derived or meat-derived products, or one or more fish or fish-derived products. The one or more dairy products can comprise whey or milk plasma, casein, milk, cheese, cream cheese, cottage cheese, yogurt or sour cream, or any combination thereof. The one or more dairy products also can comprise LIPITEIN®. The plant-based or a plant-derived organic matter can comprise a vegetable oil, a waste vegetable oil (WVO), a straight vegetable oil (SVO) or a pure plant oil (PPO).

In alliterative embodiments of the food or a feed, or a food or feed additive or supplement, or nutritional or dietary supplement, the processed organic matter in the food or a feed comprises:

(a) a rice; flaxseed (linseed), rapeseed (canola), sunflower, cottonseed, peanut, wheat, soy, barley, sorghum, oats, potato or corn plant, product or extract, or any combination thereof;

(b) a plant, product or extract from a species from a genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Phormium, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, or Zea, or any combination thereof;

(c) a plant, product or extract from a cotton, a silk cotton tree (Kapok, Ceiba pentandra), desert willow, a creosote bush, a winterfat, a balsa, a ramie, a kenaf, a hemp, a roselle, a jute, a sisal abaca, a flax, a member of the Linaceae family, a Linum usitatissimum, a Phormium tenax or a Phormium cookianum or any combination thereof;

(d) a composition, product or extract comprising or derived from a macroalgae, a seaweed; a red seaweed; a specie of the genus Ulva, Chlorophyta, Charophyta, Rhodophyta, Phaeophyceae, Porphyra, Gracilaria, Grateloupia, Kappaphycus or Ceramium; a green seaweed; a brown seaweed or brown algae; a kelp; a specie of the genus Laminaria; or, a Laminaria japonica;

(e) a plant; composition, product or extract comprising or derived from a halophyte, a plant or specie of the genus Salicornia or Spartina, or a Spartina alterniflora (smooth cordgrass or saltmarsh cordgrass) or a Spartina patens (saltmeadow cordgrass); or

(f) a composition, product or extract comprising or derived from a microalgae (also called phytoplankton, microphytes, planktonic algae).

Alternative embodiments of the food or a feed, or a food or feed additive or supplement, or nutritional or dietary supplement, are packaged, formulated or manufactured in the form of a tablet, capsule or softgel, and the like.

The invention provides methods for inactivating a protein-comprising infectious agent in a fish-derived or an animal-derived composition comprising processing the fish-derived or animal-derived composition using a process comprising a method of the invention, wherein at least one step of the process comprises: (a) raising the pH to at least pH 10.2; (b) raising the pH to between about pH 10.2 and pH 11.5; or, (c) the method of (a) or (b), and wherein at least one step of the process comprises-raising the temperature to between about 140° C. and 180° C. In alternative embodiments the protein-comprising infectious agent comprises a prion. In alternative embodiments, the protein-comprising infectious agent and/or prion is associated with or causative of a Transmissible Spongiform Encephalopathy (TSE), a scrapie, a Chronic Wasting Disease (CWD), Bovine Spongiform Encephalopathy (BSE), a Mad-Cow Disease (MCD), a Creutzfeldt-Jakob disease (CJD), a Transmissible Mink Encephalopathy (TME) or an ungulate encephalopathy.

In alternative embodiments, the methods (processes) of the invention further comprise one or steps comprising adding food supplements, nutrients, minerals or vitamins and the like to the production process. These additional ingredients, e.g., the food supplements, nutrients, minerals or vitamins and the like, can be added at any step in the process, before the process begins (i.e., to the one of the starting materials solutions or liquids, including liquids, semisolids, pastes or emulsions) or to a finished product of a process of the invention. Thus, in alternative embodiments, any product of the invention can further comprise one or more food supplements, nutrients, minerals or vitamins and the like.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

FIG. 1 graphically illustrates beef brain titration with alkali, as discussed in detail, below.

FIG. 2 and FIG. 3 illustrates an exemplary process of the invention comprising processing flow for TSE inactivation, including a “solubilization phase” (FIG. 2) and a hydroshear phase (FIG. 3), as discussed in detail, below.

FIG. 4 and FIG. 5 graphically illustrate data of recorded operating pressures for an exemplary method of this invention:

FIG. 4 (permeate mls/minute (min), and

FIG. 5 (feed mls/min), as discussed in detail in Example 1, below.

FIG. 6, FIG. 7, FIG. 8, FIG. 9, FIG. 10 and FIG. 11 graphically illustrate data of titration curve graph data points: exemplary encapsulation titration curve graph data points are illustrated in FIG. 6 (showing an encapsulation titration curve graph for fluid skim milk), FIG. 7 (showing a hydroxide titration curve graph for fluid skim milk), FIG. 8 (showing an acid titration curve graph for fluid skim milk), FIG. 9 (showing a whey protein isolate hydroxide titration curve graph), FIG. 10 (showing a whey protein isolate acid titration curve graph), and FIG. 11 (showing a soy protein concentrate hydroxide and acid titration curve graph), as discussed in detail in Example 6, below.

Like reference symbols in the various drawings indicate like elements.

### DETAILED DESCRIPTION

The invention provides methods for making and processing foods, food supplements or additives, feeds and biofuels from organic matter, e.g., any biomass, including any algae, microorganism, plant or animal. In one aspect, the invention provides methods for manufacturing, processing and recovering organic matter from a biofluid or biosemisolid comprising a protein and a lipid, which can be derived from any algae, microorganism, plant or animal. In one aspect, the invention provides methods for encapsulating oils with proteins for, e.g., making foods, food supplements or additives and feeds. Compositions made by processes of this invention can be used for foods, food supplements or additives, feeds and/or biofuels.

The invention provides methods for processing organic matter from biomass; e.g., a biofluid or biosemisolid having high moisture content and that comprise protein and lipids, where in alternative embodiments the methods of the invention encapsulate the lipid with protein in a manner that insures complete encapsulation of the lipid. For example, in one embodiment, a method of the invention comprises an encapsulation process comprising treating a protein and a lipid with an alkali, which causes the pH to be raised to a point where the protein Structure is changed such that it can “wrap around” a fat or lipid droplet, e.g., a plant, an algae-, vegetable-, animal- or fish-derived oil (e.g., create an inside out liposome or vehicle, where the oil, lipid or fat is within the encapsidation product, or “droplet”, where the oil, lipid or fat is surrounded by protein, where the protein is in contact with solvent, e.g., water).

In one embodiment, the invention provides methods for making human foods, food supplements or additives, or animal feeds by encapsulating an oil, e.g., a microorganism- algae-, fish-, animal- or plant-derived oil, in a protein, e.g., a plant- or animal-derived protein; and some of these embodiments are “heart-friendly” foods and food supplements or additives, for example, fish oils or flax oils encapsulated in a plant-derived protein such as a soy protein or a dairy-derived protein such as a casein or a whey. Alternatively, the oil can comprise a plant-based or a plant-derived oil such as a vegetable oil, a waste vegetable oil (WVO), a straight vegetable oil (SVO) or a pure plant oil (PPO).

In alternative embodiments, processes of the invention provide methods to separate all, some, substantially most of or the majority of moisture from a protein-encapsulated lipid; where this separation can be by drying, vacuum drying, and the like.

### Organic Matter Starting Materials

In one embodiment, the invention provides methods for processing and recovering organic matter from a biofluid, biosemisolid or biosolid (e.g., any biomass) comprising a protein and/or a lipid. In an alternative embodiment, the invention provides processes for producing foods, food supplements or additives, feeds and biofuels from organic matter in a commercially viable manner. The invention also provides foods, food supplements or additives, feeds and biofuels made by the processes of this invention.

In alternative embodiments, organic starting materials for the processes of this invention and the compositions of the invention (e.g., foods, food supplements or additives, animal feeds and biofuels) can comprise or be derived from any plant, animal, microorganism and/or algae; e.g., including animal meat or blood, milk or milk-based products (e.g., casein or whey), by-products or wastes, fish products, by-products or wastes, microbes, food supplements or additives, food (e.g., flax or soy) or biofuel crops, algae, and/or waste-streams, e.g., from abattoir waste-stream proteins.

Organic starting materials for the processes of this invention may contain undesirable solids, for example abattoir waste stream proteins are likely to contain ungular (e.g., a hoof, claw or talon) parts. Thus, in alternative embodiments, the methods can comprise a solubilization and/or crude grinding step of organic matter to reduce the size of solids, e.g., the undesirable solids. The grinding can be accomplished using any conventional chopping mechanism; e.g., any chopping pump such as a chopping trash pump, such as the “Parma Chopper Pump” made by Parma Co. (Parma, Id.). In the case of processing materials such as fish waste, the chopping mechanism (e.g., chopping pump) will break the undesirable solids up to the point where they can be pumped (e.g., by conventional or non-chopping pumps).

In one aspect, e.g., where a starting protein source comprises a blood or other any protein source in which the particle size of undesirable solids are initially small enough to pass through a (non-chopping) pump, the chopping mechanism (e.g., chopping pump or chopping trash pump) may not be necessary. In alternative embodiments, a screen can be used to remove undesirable solids either before, during and/or after passing through a chopping mechanism, or as a substitute for using a chopping mechanism.

In addition to the “Parma Chopper Pump”, other exemplary pumps that can be used to practice this invention, e.g., for handling raw materials (e.g., unprocessed or partially processed organic matter), include: a Vaughan Chopper Pump®, and Triton™ screw centrifugal pump and/or a Vaughan Co., Inc., Montesano, Wash., USA.

### Manipulating pH Levels

In alternative embodiments, the processes of the invention comprise adjusting the acidity and alkalinity (pH) of the organic starting materials and intermediary materials for the processes of this invention, e.g., to generate a precipitate or a flocculate (by raising pH, or alkalinizing), or to solubilize or denature a protein or protein-lipid composition (by lowering pH, or acidifying).

Alkalizing or Raising pH

In alternative embodiments methods of the invention comprise adding to a first liquid and/or semisolid a second liquid comprising a base (e.g., an alkali) to raise (make more alkaline) the pH of the first and second liquid mixture. In alternative embodiments, the pH is adjusted to about pH 8 and about pH 13, or between about pH 11 and about pH 12, or about pH 8, about pH 9, about pH 10, about pH 11, about pH 12 or about pH 13. In one embodiment the pH is adjusted (raised) to a point at which the protein flocculates or coagulates, or the protein and lipid form an emulsion. In one embodiment the pH is adjusted (raised) to a point at which the protein and lipid form a lipid-encapsulated structure (a liposome or vesicle comprising the protein) or a protein-encapsulated structure.

In one embodiment, methods of the invention-precisely manipulate pH levels to optimize capsule strength and durability and minimize the quantity of exogenous chemical use; for example, see Example 6, below. The optimum pH of solubilization can be determined by titration for each proteinaceous mixture to be used because the solubilization pH range varies depending on protein type. In general, these titrations are determined with samples of the target proteinaceous material and measured in the laboratory to determine the optimum pH of solubilization. The pH points can then be used during commercial production. Optimum pH of solubilization can be determined as an alkali hydrogen ion difference on a graph in which rate of change in hydrogen ion concentration varies with acid equivalent units, e.g., as described in U.S. Pat. No. 5,514,388.

For example, in one aspect, an organic material (e.g., an organic starting material) for a process of this invention, e.g., a protein-lipid mixture or a proteinaceous mixture, can be titrated with an alkali. Rate of change of hydrogen ion difference can be measured and when the slope of the titration curve is essentially zero, then addition of alkali is ceased. In alternative embodiments, “acid equivalent units” means the base addition data is corrected to the acid unit and the term “base equivalent units” means the acid addition data is corrected to the base unit; the term “alkali hydrogen ion difference” means the absolute difference in hydrogen ion concentration per base equivalent unit; the term “acid hydrogen ion difference” means the absolute difference in hydrogen ion concentration per acid equivalent unit.

In one aspect, solubilization is accomplished by introducing an alkali source (e.g., a calcium hydroxide, or a Ca(OH)₂ or slaked lime or hydrated lime, a sodium hydroxide or lye or caustic soda, a potassium hydroxide, a calcium oxide or a magnesium hydroxide) into a vat or tank in which the temperature is being controlled via: a water jacket, or oil heated jackets; on a vat, a tank, a liquid storage unit or other suitable means. The water or oil jacket can be designed with internal baffles or equivalent to insure uniform heating of the tank, vat, etc. An organic material (e.g., a protein-lipid mixture or a proteinaceous mixture) or a liquid used as part of a process of this invention can be, continuously re-circulated through the tank, vat, etc.

In one embodiment, the organic material (e.g., protein-lipid or proteinaceous mixture) is maintained at a temperature above the melting point of a lipid to be used (e.g., a lipid in the organic material to be processed); the optimum temperature can be between about 50° C. to 55° C., depending on the type and amount of lipid(s) in the organic material to be processed.

In one embodiment, additional lipid or lipids (e.g., fats) can be added for “encapsulation” with protein in the starting material. Lipid may be added at any time in the process, e.g., before, during or following solubilization and/or mixing, shearing or sonicating the alkalinized liquid staring organic material. In one embodiment, if the lipid is added following solubilization, then any drop in pH due to free fatty acids is corrected at least back to the optimum pH of solubilization.

The selection of one or a mixture of the hydroxides of calcium, magnesium, sodium and potassium and/or the carbonates of sodium and potassium provide(s) the alkali and allow(s) control of the amount and ratios of the cations found in the finished product.

By the proper selection of the alkali(s) and acid(s) used, the nutritional and electrolyte value of the finished product can be modified. Weak acids also provide additional antimicrobial protection.

Acidifying or Lowering pH

In alternative embodiments methods of the invention comprise adding a liquid comprising an acid, e.g., added in a mixing step, where the liquid comprises a phosphoric acid, a nitric acid, a boric acid, a hydrochloric acid, a sulfuric acid, a lactic acid, an acetic acid, a propionic acid (propanoic acid), a formic acid, a citric acid, an oxalic acid, or an organic acid, or any combination thereof. In one embodiment, methods of the invention comprise mixing, shearing or sonicating a liquid and at the same time or any time during the process of the mixing, shearing or sonicating, adding a liquid comprising an acid.

Optimum pH can be determined by titration for each organic material, e.g., lipid-proteinaceous mixture to be used; as the encapsulation pH range varies depending on the types of proteins, lipids etc are in the starting material. Titrations can be determined with samples of the target organic (e.g., proteinaceous) material and measured in the laboratory to determine the optimum pH, e.g., optimum pH or encapsulation, or optimum pH for generating a precipitate or flocculant comprising substantially most of the protein, or optimum pH for having lipid or lipid-encapsulated structures or protein-encapsulated structures fall out of the liquid, or optimum pH for the point where substantially most of the protein, lipid, or lipid-encapsulated structures or protein-encapsulated structures precipitate or flocculate out of the liquid. These pH points can then used during commercial production.

Optimum pH can be determined as an acid hydrogen ion difference on a graph in which rate of change in hydrogen ion concentration varies with acid equivalent units, e.g., as described in U.S. Pat. No. 5,514,388. For example, the liquid mixture can be titrated with acid and the rate of change of hydrogen ion difference can be measured; and when the slope of the titration curve is essentially zero, then addition of acid is ceased. If it is desired to make lipid-protein encapsulations, capsules can be created in a range at or below the pH of encapsulation, but using less than the amount of acid which would partially or completely break down the primary structure of the protein. This pH range inhibits growth of bacteria and other harmful micro-organisms and results in a dry encapsulated lipid, safe for use as a human food, food supplement and/or nutritional or dietary aid, and/or an animal feed, supplement and/or nutritional or dietary aid.

The acid can added using a mixer, e.g., a mixer with substantially propeller-shaped blades, or any device that produces gentle fluid turbulence at the point of addition will accomplish the smooth addition of the acid. An alternative embodiment comprises adding the acid immediately following one or more inline shearing or mixing device or devices, e.g., a HYDRASHEAR™ device (Parma Laboratories, Inc., Parma Idaho). In one aspect, the acid is injected into to the center of the stream flowing past the one or more shearing or mixing devices; e.g., a HYDRASHEAR™ device(s), into the turbulence immediately following the shear. In alternative embodiments, after processing with a shearing or mixing device, e.g., a HYDRASHEAR™ device, the encapsidation product (e.g., protein surrounding lipid, or vice versa) is reduced to a size of between about 10 to about 50 microns, or about 5, 10, 15, 20; 25, 30, 35, 40, 45, 40, 50, 55 or 60 or more microns.

The selection of one or a mixture of acids used to lower the pH allow(s) control of the ratios and amounts of phosphate; chloride and sulfate, the ratios and amounts of anions from the strong acids (phosphoric, hydrochloric and sulfuric) and from the weak acids (formic, acetic and propionic) and the ratios and amounts of formic, acetic and propionic in the finished product.

In one embodiment, a pump for feeding the HYDRASHEAR™ device for the encapsulation step is a MOYNO®Sanitary High Pressure Pump, Moyno, Inc., Springfield, Ohio, USA.

### Filtering

The organic starting material, e.g., a protein/lipid blend or mixture, can be filtered before or during any step of a process of this invention, e.g., before, during or after adding to a first liquid and/or semisolid a second liquid comprising a base to raise the pH of the mixture to a point at which the protein flocculates or coagulates, or to raise the pH of the mixture to a point at which the protein and lipid form an emulsion, or to raise the pH of the mixture to a point at which the protein and lipid form a lipid-encapsulated structure (a liposome or vesicle comprising the protein) or a protein-encapsulated structure. Any effective standard filtering method can be used, e.g., any standard filtering methods can be used, for example, pumping the organic material-comprising liquid past a conventional screening device such as a screen (e.g., from Sweco, Florence, Ky.), a vibrating screen, a rotating screen or a cloth filters. In alternative embodiments, it may be desirable use one or more filters or filter steps when processes of the invention use as an organic, material a source (e.g., a protein/lipid stream) having undesirable solid materials, including e.g., dirt, rocks, glass, metal, hair, bone, feathers, plant fiber, etc. For example, methods of the invention can process plant waste streams, or food, fish or animal processing plant waste streams, or human, fish, poultry (fowl, e.g., chicken) or animal e.g., pig) excrement (sewage) processing plant waste streams. Filtering can allow the removal of undesirable solid materials and difficult to solubilize protein sources, for example hair, feathers, horn or hoofs. Additionally, filtering can allow the removal of undesirable fibrous materials from plant sources of protein, allowing concentration of the protein. If the fibrous materials are not filtered out of the liquid mixture fibers may provide a source of physical and mechanical obstruction, e.g., prevent good encapsulation of a lipid by a solubilized organic matter (e.g., proteinaceous) mixture. In one aspect, filtering removes materials which would not pass the shearing and mixing device and would contaminate the final product.

### Shearing and Mixing

In alternative embodiments, processes of the invention comprise mixing, shearing and/or sonicating the alkalinized liquid of a previous step and at the same time or any time during the process of the mixing, shearing or sonicating, adding a liquid comprising an acid to: lower the pH (make the liquid more acid) to between about pH 3 and pH 6, or about pH 3, about pH 4, about pH 5, or about pH 6, thereby generating a precipitate or flocculant comprising substantially most of the protein, lipid or lipid-encapsulated structures or protein encapsulated structures out of the liquid, or lower the pH (make the liquid more acid) to the point where substantially most of the protein, lipid, or lipid-encapsulated structures or protein-encapsulated structures precipitate or flocculate out of the liquid.

In one aspect, shearing and mixing is done in such a way that no air is entrained in the emulsion and complete shearing occurs; in some applications this allows complete encapsulation of the lipid materials, which may facilitate large-scale commercial production.

In one embodiment, the protein/lipid mixture or blend is pumped into a cone bottom tank surrounded by a hot water jacket. In one aspect, the cone of the tank is not jacketed to prevent excessive heating and possible charring of the tank contents. The bottom of the cone can be designed in a manner to insure a continuous supply of the thick gelled product to the inlet side of the pump. The protein/lipid mixture or blend (e.g., the proteinaceous mixture/lipid blend) can be pumped past a shearing and/or mixing device, e.g., a HYDRASHEAR™, device. In one embodiment, pump delivers the material to the HYDRASHEAR™ device or other shearing and/or mixing device with a line pressure above 25 pounds (pound-force) per square inch (psi), or in the range of between about 60 to 150 psi, or between about 150 to 240 psi, or at about 60, 70, 80, 90, 100, 110, 120, 130, 140, 150, 160, 170, 180, 190, 200, 210, 220, 230 or 240 or higher psi. The discharge of the pump can be plumbed to the shearing and/or mixing device and, in one embodiment, immediately thereafter an in-line injector is used to add acid (e.g. in one embodiment the process of the invention comprises mixing, shearing or sonicating an alkalinized liquid and at the same time or any time during the process of the mixing, shearing or sonicating, adding a liquid comprising an acid).

The HYDRASHEAR™ causes formation of lipid droplets surrounded by protein. The design of the HYDRASHEAR™ allows formation of the protein/lipid emulsion without the entrainment of air, and in a manner such that protein/lipid droplet size is very small. This equipment also allows the addition of acids or other denaturants without the entrainment of air, before lipid droplets can reform into increasingly larger droplets. Some heat is generated due to friction from pumping the material past the HYDRASHEAR™. Other mixers which allow formation of protein/lipid emulsions without entrainment of air and in a manner such that protein/lipid droplet size is very small can also be used.

In one embodiment, plumbing returns the product to a vat, a tank (or whatever other suitable storage unit is used) and discharges it in the lower portion of the vat, tank or storage unit through aspirators, mixing nozzles or mixing eductors (e.g., Fox Venturi Eductors/Fox Valve, Dover, N.J.). The aspirators, mixing nozzles or mixing eductors can be arranged in a manner which maximizes mixing while preventing the entrainment of air. In one aspect, the product can be diverted to a dryer or to an intermediate storage before drying. In another aspect, the product is sent or diverted for further separation, e.g., on a screen (e.g., a vacuum screen), a membrane or by centrifugation; and alternatively, dried after these further separation steps.

Additional Dilution Steps

In alternative embodiments, initial (unprocessed) organic matter, compositions from intermediate steps of the methods of this invention, of final product compositions (including liquids, semisolids and pastes, and/or solids) are diluted and/or further hydrated before initial or further processing, or packaging if in the form of a final product (e.g., a food, feed, supplement, additive, dietary aid or biofuel and the like). Intermediate liquids, semisolids (e.g., pastes) and/or solids can be diluted and/or further hydrated with water, milk, blood, plant oil, algae oil, animal oil and the like.

For example, in one aspect, soy protein and blood meal (dried, powdered blood) and/or bone meal are diluted before isolation after acid precipitation or flocculation, or after a separating or removing step (e.g., the step of separating or removing some, all or substantially most of the liquid from the precipitate or flocculate, or separating or removing some, all or substantially most of the precipitate or flocculate from the liquid).

The dilution or hydration step can facilitate further processing of protein, lipid, or protein and lipid form a lipid-encapsulated structures (liposomes or vesicles comprising the lipid encapsulating the protein) or a protein-encapsulated structures (liposomes or vesicles comprising a protein encapsulating a lipid), e.g., by separation on a screen (e.g., isolation by pulling down over a vacuum screen), a membrane and the like.

Alternatively, the dilution or hydration step can be simply for mixing different compositions from different sources, e.g., from diary and plant sources. In alternative embodiments, the dilution or hydration step can be for adding in additional ingredients, as discussed below, e.g., including enzymes, nutrients such as liquid or fat soluble nutrients, vitamins, ionophores, phytogenics, synbiotics, carbohydrates, prebiotics, fats (e.g., essential fatty acids (EFAs) such as ω-3 (or omega-3 or n-3) and ω-6 (omega-6, n-6) fatty acids, or linoleic acid, and the like.

For example, in some embodiments of the processes of the invention it is desired to have about 15%, or between about 10% to 25%, dry matter to give optimal viscosity for an intermediary or a finished product. For example, in one aspect extra fluid (e.g., water, milk) is added before, during and/or after the alkalinizing step; where in some aspects a viscosity of a composition having between about 10% to 25%, dry matter is desired or optimal for further processing, e.g., for the acid coagulation or precipitation step, or a final separating or removing step by e.g., vacuum separation (e.g., on a screen or membrane), filtering and/or centrifugation. In some embodiments, liquid-diluted lipid or fat is added to a protein-comprising organic matter before the alkalinizing step where at which the protein flocculates or coagulates, or the protein and lipid form an emulsion or encapsulated structure (e.g., a vesicle). In the case of a hydrate final product, a resulting “dry paste” can be added to a food (e.g., a yogurt) or a feed (e.g., as a cattle, or calf food).

Further Isolation of Proteins and Lipids from Initial Treated Material

In alternative embodiments, the methods of the invention also comprising treating the liquid or Semisolid after the separation step (e.g., separating or removing some, all or substantially most of the liquid from the precipitate or flocculate, or separating or removing some, all or substantially most of the precipitate or flocculate from the liquid) from which the precipitate or flocculate has been separated/removed by an additional protein and/or lipid separation procedure. The additional step removes/separates protein and/or lipid (and other solids in some aspects) not separated or removed by the initial separation step.

In some aspects, this optional “further refinement” or purification of the liquid makes (processes) the liquid to a form more acceptable for disposal in the environment, e.g., into a public sewage system. Alternatively, this additional step generates additional solids, including protein, lipids or protein-encapsulated lipids, for addition to, e.g., a food, supplement, additive, dietary supplement, feed or biofuel. Similarly, the “further treated” liquid, depending on the desired application, can be more suited for use in or with a food, supplement, additive, dietary supplement, feed, or biofuel, and the like. Alternatively, the solids, lipids and/or proteins separated by this “additional” step can be added back to the initial precipitate or flocculate.

In alternative embodiments, the additional protein and/or lipid separation procedure comprises precipitation, centrifugation, filtering, chromatography, electrophoresis, or a combination thereof. The filtering can comprise filtering with a screen or vacuum screen, a membrane, or filtering by ultrafiltration or nanofiltration. The filtering with a membrane can comprise filtering with a polyvinylidene fluoride (PVDF) membrane (e.g., a UF-PVDF-30K membrane) or a microporous membrane, or any combination thereof.

In one aspect, the microporous membrane comprises a pore size ranging between about 0.1 to 10 micrometers (μm). The ultrafiltration can comprise filtering with a selectively-permeable membrane, a partially-permeable membrane or a differentially-permeable membrane, or any combination thereof. The ultrafiltration can comprises filtering with a cellulose ester membrane (CEM), a charge mosaic membrane (CMM), a bipolar membrane (BPM), an anion exchange-membrane (AEM), an alkali anion exchange membrane (AAEM) or a proton exchange membrane (PEM), or any combination thereof. The precipitation can comprise precipitating protein not separated or removed by a separation, e.g., with an ammonium sulfate ((NH₄)₂SO₄) solution, or equivalent. The centrifugation can comprise a density gradient centrifugation, an equilibrium centrifugation or a non-equilibrium centrifugation, or any combination thereof. The chromatography can comprise a size exclusion chromatography, an ion exchange chromatography; an affinity chromatography, a high performance liquid chromatography (HPLC) or a high pressure liquid chromatography, or any combination thereof. The electrophoresis can comprise a denaturing-condition electrophoresis or a non-denaturing-condition electrophoresis, or any combination thereof.

In alternative embodiments, separation equipment (e.g., for separating or removing some, all or substantially most of the liquid from the precipitate or flocculate, or separating or removing some, all or substantially most of the precipitate or flocculate from the liquid) that can be used to practice any step of the invention include e.g., a Model SC-3536-177 liquid/liquid separator; a Westfalia Separator, Northvale, N.J., USA; an AFPX solid-ejecting centrifuge, Alfa Laval, Inc., Lund, Sweden; a Dorr-Oliver horizontal belt filter, a FLSmidth Dorr-Oliver Eimco, Salt Lake City, Utah, USA; and/or a Rotary vacuum filter, WesTech Engineering, Inc., Salt Lake City, Utah, USA.

### Drying and Concentration of Product

In alternative embodiments, the processes of the invention comprise a step comprising partially or completely drying proteins, lipids and/or solids not initially separated/removed by an initial separation, but rather separated/removed in subsequence step, e.g., by an additional protein and/or lipid separation procedure comprising precipitation, centrifugation, filtering, chromatography, electrophoresis, or a combination thereof. In one embodiment, the process comprises drying proteins, lipids and/or solids not separated or removed when an initial precipitate or flocculate is separated or removed from a lowered pH (acidified) liquid.

In alternative embodiments, the partial or complete drying comprises lyophilization (freeze-drying or cryodesiccation), vacuum drying, vacuum kiln drying, evaporation, application of heated air (convective or direct drying), indirect, or contact drying (heating through a hot wall), drum drying, supercritical drying (superheated steam drying), spray drying, natural air (drying by treating with unheated forced air), or any combination thereof.

In alternative embodiments, the protein, precipitate or flocculate whether partially or completely dried or not, is concentrated and/or further dried by lyophilization (freeze-drying or cryodesiccation), ultrafiltration, or any combination thereof.

In alternative embodiments, processes of this invention do not use or add additional liquids to the initial organic starting material; e.g., processes of this invention do not add extraneous water to the liquid mixtures, e.g., to dilute or solubilize the organic matter (e.g., protein, lipid or protein-lipid mixtures).

Final product or material made by a process of this invention can be formed as a granule, a flakes or a powder, depending e.g. on the drying method used. Final product or material made by a process of this invention also may be liquid form without drying, or concentrated liquid form with partial drying. In one embodiment, because the lipid is protected from oxidation by the surrounding protein, final product or material made by a process of this invention has an extended shelf life and is non-greasy, both of which make it easily handled and transported.

In alternative embodiments, drying equipment that can be used to practice this invention includes e.g., a Double drum dryer, Buflovak, Inc. Buffalo, N.Y., USA; a Niro Vibro-Eluidizer® two-stage spray dryer, Niro Inc., Hudson, Wis., USA; and/or a Barr-Rosin Ring Dryer, GEA Barr-Rosin (USA) Inc., St. Charles, Ill., USA.

### Additional Ingredients

Additional ingredients can be added to the initial organic material or any intermediary liquid, semisolid or solid of a method of this invention, or to an intermediate or final (completed) composition made by a method of this invention. These additional ingredients can be added for the purpose of making foods or feeds, food or feed additives or supplements (see below) and the like.

In alternative embodiments, the methods (processes) of the invention further comprise one or steps comprising adding additional materials, e.g., for health (including prophylactic or preventative reasons), medicinal or nutritional reasons, as a digestive aid, for flavoring, for coloring, as a preservative, for texture (e.g., “smoothness”), etc.; the additional materials that can be added include for example: enzymes, food supplements, antioxidants (e.g., vitamin C or vitamin E, or glutathione) nutrients, minerals or vitamins (e.g., a vitamin B, such as niacin), an ionophore, a phytogenic, a synbiotic, chelating agents (e.g., EDTA, or ethylenediaminetetraacetic acid) and the like (e.g., see below) to the production process. These additional ingredients, e.g., the food or feed supplements, nutrients, minerals or vitamins and the like, can be added at any step in the process (e.g., during the process), before the process begins (i.e., to the one of the starting materials solutions or liquids, including liquids, semisolids, pastes or emulsions) or to a finished product of a process of the invention. Thus, in alternative embodiments, any product of the invention can further comprise one or more food or feed supplements, nutrients, minerals or vitamins and the like. The additional ingredients can be for man or animal (including e.g., mammal or fish), e.g., for supplementation of foods or feeds, as food or feed supplements or additives, and the like.

For example, additional ingredients include enzymes (see below), nutrients such as liquid or fat soluble nutrients, a composition acting as a rich nitrogen source (e.g., protein concentrates, or bone or blood meal), vitamins (e.g., biotin, choline or any B vitamin such as niacin), an ionophore such as monensin (e.g., RUMENSIN™) or lasalocid (BOVATEC™), a phytogenic (a substance derived from a herb, spice or aromatic plant having antimicrobial, antifungal, antiviral, antioxidant or sedative properties), a synbiotic (combined administration of probiotics and prebiotics), a carbohydrate (e.g., fructose, xylose, glucose) or any “prebiotic”, a fat or lipid (e.g., an essential fatty acid (EFA) such as ω-3 (or omega-3 or n-3) and ω-6 (omega-6, n-6) fatty acids, or linoleic acid, alpha linolenic acid or arachidonic acid), or an n-9 fatty acid (also called ω-9 fatty acids or omega-9 fatty acids, such as oleic acid), an amino acid (e.g., methinione) or a powdered or concentrated protein (e.g., soy powder), a salt (e.g., sodium chloride or potassium chloride), a mineral (e.g., iron, zinc), zootechnical additives (e.g. digestibility enhancers, gut flora stabilizers), an antibiotic or other antimicrobial (e.g., coccidiostats or histomonostats), a drug such as a steroid, or any steroid or polypeptide, e.g., for use as a growth regulator, e.g., as a growth hormone; and/or an immune stimulant (e.g., a feed additive that functions as a stimulator or modulator of the immunity processes, e.g., a cell wall fragments from a bacteria or a yeast or a sea algae to induce activation of an immune cell). Enzymes, e.g., a protease, phytase (e.g., PHYZYME XP® phytase, Verenium Corporation, San Diego, Calif.), xylanase, lipase, amylase and the like also can be added, e.g., into a starting material, in the middle of the process, or to a final product produced by a process of this invention.

In one embodiment, concentrated protein compositions, e.g., an amino acid concentrate, or a powdered or concentrated protein such as a soy powder or soy concentrate, bone meal or blood meal is added in powdered or liquid form.

For example, nutrients such as liquid or fat soluble nutrients, such as vitamins, can be included or added any time during a process of the invention, or at the beginning or end of a process (e.g., to a starting material or finished product of a process of the invention). In one embodiment, if a fat susceptible to oxidation (susceptible to rancidity) is in the initial organic matter processed by a method of the invention, or is added in e.g., as a “fat soluble nutrient”, an antioxidant, for example: a vitamin E or an ethoxyquin, a vitamin C, a tocopherol, BHA, BHT also can be included or added any time during a process of the invention.

Technological additives also can be added to the initial organic material or any intermediary liquid, semisolid or solid of a method of this invention, or to an intermediate or final (completed) composition made by a method of this invention, at any time during the process. Technological additives include e.g. coloring agents, flavoring agents, preservatives, antioxidants, emulsifiers, stabilizing agents, acidity regulators, silage additives: and the like.

Probiotics also can be added to the initial organic material or any intermediary liquid, semisolid or solid of a method of this invention, or to an intermediate or final (completed) composition made by a method of this invention, at any time during the process; e.g., to the final completed product. Probiotics that can be added include live microorganisms or viable spores which support the development of a beneficial gut microflora, e.g., probiotic bacteria from the genera Lactobacillus, Bifidobacterium, Enterococcus that counteract undesired microorganisms such as Salmonella or E. coli by blocking receptors on the gut wall, production of antimicrobial substances or activation of the immune system.

Neutral fillers (“roughage”) or dietary fibers, e.g., fibrous material, also can be added to the initial organic material or any intermediary liquid, semisolid or solid of a method of this invention, or to an intermediate or final (completed) composition made by a method of this invention, at any time during the process. For example, neutral fillers or dietary fibers that can be added include a plant pulp, a seed husk or a hull, e.g., soybean, corn or sunflower husks or hulls, a beet or sugar beet pulp, bagasse, and the like.

Foods and Feeds, and Food and Feed Additives and Supplements

The invention provides methods for making foods (including solids or liquids) and feed (including solids or liquids), food and feed supplements and food and feed additives (including solids or liquids), e.g., nutritional or dietary supplements (including solids or liquids), and the like, and compositions (foods, feeds, additives, supplements, nutritional aids) made by the methods of this invention. In one embodiment, the invention provides methods for making animal or human foods, food supplements or additives, or animal feeds, or feed supplements or additives, by encapsulating an oil, e.g., a microorganism-algae-, fish-, animal- or plant-derived oil, in a protein, e.g., a plant- or animal-derived protein.

In alternative embodiments, foods, food additives or supplements, and nutritional aids are “heart-friendly”. For example, in alternative embodiments of methods and compositions of the invention fish oils or flax oils (e.g., linseed oil, also known as “flax seed oil”) are encapsulated in an algae- or plant-derived protein, such as a soy protein, or a dairy-derived protein, such as whey or casein. In alternative embodiments, any oil, e.g., an oil comprising omega-3 fatty acids, eicosapentaenoic acid (EPA) and/or docosahexaenoic acid (DHA) and the like, is used in a process or composition of this invention, e.g., including oils from fish such as mackerel, lake trout, flounder, tuna (e.g., albacore tuna) and salmon, or oils from algae, e.g., microalgae or microphytes.

In alternative embodiments, additional oils or fats are added to the extent needed to mimic the fat content of an already marketed food or liquid. For example, consumers are comfortable and used to consuming milk having a fat content of between about 1% to 2% to 3.25%. Thus, oils or fats are added in alternative embodiments of the invention such that the final product has a final total fat or oil concentration of at about the level of fat free milk or skim milk, 1% milk (1% fat), 2% milk (2% fat), 3.25% (whole milk) to 3.5% (the fat level for standard milk for trade between the dairy and milk processor); but for a particular purpose also can be more. In one embodiment, the fat or oil content is set to between about 3% to 3.5%, this range is desirable for this embodiment if the omega 3 fats are at adequate levels according to the American Heart Association suggested guidelines. The fat or oil content can be any mixture of both plant (e.g., flax oil) and animal fats or oils.

In alternative embodiments, additional ingredients added to a processor a product of the invention include calcium and calcium-comprising compositions, solids, powers and/or liquids. In alternative embodiments, additional ingredients are “food grade” or USP grade. For example, in alternative embodiments additional calcium oxide and/or calcium hydroxide is added, which can be USP or food grade, e.g., ingestible-grade material. This “additional ingredient” calcium is in addition to the calcium that might be in a base added in a step of a process of the invention, e.g., in the form of a calcium hydroxide, or a Ca(OH)₂ (or slaked lime or hydrated lime), a sodium hydroxide (lye or caustic soda), a potassium hydroxide or a calcium oxide. In alternative embodiments, the additional ingredient is added for health reasons, e.g., as a nutritional supplement for increasing bone density or strength, e.g., to treat osteoporosis. Other minerals, e.g., iodine, iron (e.g., for anemia), potassium, phosphorus or phosphate, chloride, magnesium, molybdenum, zinc, selenium and the like can also be added. For example, additional phosphorus or phosphate is added as “food grade” or USP grade acid, e.g., phosphoric acid, or any “ingestible acid.”

In alternative embodiments, additional ingredients added to a process or a product of the invention include vitamins, including water and fat soluble vitamins. For example, any of the following water-soluble B vitamins can be added:
Vitamin B₁ (thiamine)
Vitamin B₂ (riboflavin)
Vitamin B₃ (niacin or niacinamide, sometimes also known as vitamin PP; or inositol niacinate, or inositol hexanicotinate).
Vitamin B₅ (pantothenic acid)
Vitamin B₆ (pyridoxine, pyridoxal, or pyridoxamine, or pyridoxine hydrochloride)
Vitamin B₇ (biotin)
Vitamin B₉ (folic acid)
Vitamin B₂ (various cobalamins; commonly cyanocobalamin in vitamin supplements)
Vitamin C or L-ascorbic acid is another water-soluble that can be added; it can be added as an antioxidant. Vitamin E, a fat soluble vitamin, or tocotrienols or tocopherols (e.g., α-tocopherol), also can be added, e.g., as an antioxidant. Any of the fat soluble vitamins A (e.g., retinol or any diterpene), vitamin D or any fat-soluble secosteroids (e.g., vitamin D₂ or ergocalciferol, or vitamin D₃ or cholecalciferol), vitamin E or any α-, β-, γ-, and δ-tocopherol, and/or vitamin K or any phylloquinone, can be added.

In alternative embodiments, additional ingredients can be known medicinal agents, e.g., therapeutic or prophylactic medicines or agents, painkillers, anti-inflammatories, and the like. The additional ingredients can be conventional prescription drugs or “over the counter” drugs or therapeutic, or they can be “natural” remedies or therapies, e.g., herbal medicines, palliatives and the like.

In alternative embodiments, additional ingredients can be chelation agents, e.g., for use to detoxify poisonous metal agents such as mercury, arsenic and lead.

In alternative embodiments, additional ingredients that are less than palatable to human or animal tastes (e.g., they don't taste good, such as a rancid fat or oil) are added at the beginning of a process of the invention, e.g., to a starting material (e.g., a first liquid and/or semisolid or paste comprising an organic matter or a biomass in a liquid, a semisolid or paste, a colloidal and/or a solid or a particulate form; or a second liquid comprising a base to raise the pH) or to a first step of a process of the invention, e.g., addition and/or mixing of the first liquid and/or semisolid or paste and the second liquid. This alternative embodiment has the advantage of “encapsulating” the additional ingredient or ingredients that is/are “less than palatable” in a protein, lipid, or lipid-encapsulated structures or protein-encapsulated structure made by a process of the invention. The “encapsulation” ameliorates or masks the unpalatable quality (e.g., the bad taste) of the additional ingredient.

As noted above, any ingredient can be added at one or more steps of a process of the invention, or at the beginning or end of a process: e.g., to a starting material or finished product of a process of the invention. In alternative embodiments, the “additional ingredients” (e.g., nutrients, vitamins, minerals, food additives, supplements, or nutritional aids and the like) are added to a food or feed in one or more steps or intermediary ingredients of a manufacturing process of the invention.

In one aspect, the food additives or supplements, and nutritional aids of the invention (e.g., products made by a process of this invention) are packaged as oral formulations, e.g., as capsules, tablets, “soft-gels”, e.g., a soft gelatin shell surrounding a liquid fill, for example, as a softgel shell comprising gelatin, water, and glycerin.

### Biofuels

The invention provides biofuels and synthetic fuels made using processed organic materials using methods of this invention, and using compositions made by the methods of this invention. The biofuel made using processed organic materials can be in the form of a gas, or gasoline, e.g., a syngas. In one aspect, methods of the invention comprise treating compositions made by the methods of this invention with enzymes in a natural biomass conversion process, e.g., a hydrolysis and/or fermentation of the processed organic materials, to make a biofuel, e.g., a bioethanol, biopropanol, bio-butanol or a biomethanol, or a synthetic fuel, in the form of a liquid or as a gas, such as a “syngas”.

For example, invention provides methods for making biofuel gases and synthetic gas fuels (“syngas”) comprising a bioethanol, biopropanol, bio-butanol and/or a biomethanol made using processed organic materials made using methods of this invention, and enzymes and/or fermentation. In one aspect this biofuel gas of the invention is mixed with a natural gas (can also be produced from organic material/biomass), e.g., a hydrogen or a hydrocarbon-based gas fuel.

### Inactivating Infectious Agents

The invention also provides methods for processing protein-comprising materials to inactivate infectious agents, e.g., prions. For example, in alternative embodiments the invention comprises methods for inactivating a prion protein in an animal-derived composition comprising processing the animal-derived composition using a process comprising a method of any one of claims 1 to 65, wherein at least one step of the process comprises: (a) raising the pH to at least pH 10.2; (b) raising the pH to between about pH 10.2 and pH 11.5; or, (c) the method of (a) or (b), and wherein at least one step of the process comprises raising the temperature to at least about 140° C., or between, about 140° C. and 180° C. In alternative embodiments pressure above about 9.5 atmospheres is also used (applied to the organic material to be processed by a method of the invention).

In alternative embodiments methods of the invention treat possibly infected material to these high pH (e.g., at least pH 10.2) or high pH and high temperature (at least about 140° C.) conditions, to denature, unfold and inactivate protein-comprising infectious agents, e.g. prions and viruses. In some aspects, the infectious agents partially or completely neutralized and/or inactivated (e.g., unfolded and/or denatured) by a method of this invention includes prions causative for a Transmissible Spongiform Encephalopathy (TSE) such as scrapie in sheep or Chronic Wasting Disease (CWD) in deer, Bovine Spongiform Encephalopathy (BSE), commonly known as Mad-Cow Disease (MCD), Creutzfeldt-Jakob disease (CJD) in humans, Transmissible Mink Encephalopathy (TME), ungulate encephalopathy, and the like.

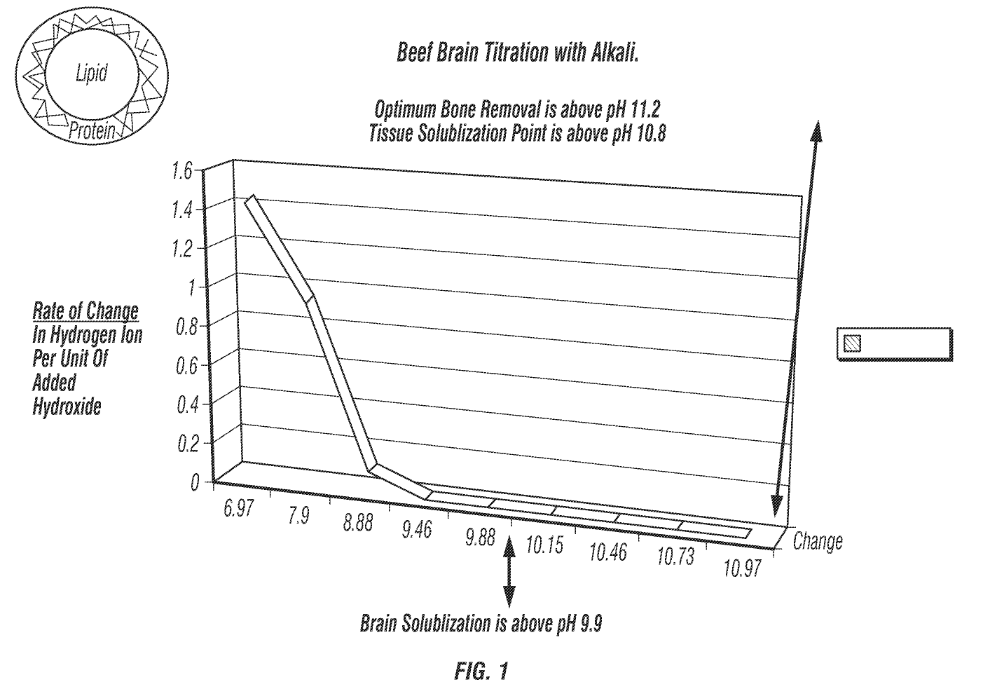

For example, FIG. 1 graphically illustrates beef brain titration with alkali, such titrations can aid in designing the pH profile of any protocol of the invention depending on the composition of the starting material, e.g., beef brain in this example.

TSE's occur when the prions show an increase of beta sheet structure and a decrease in alpha helix structure. In one embodiment, methods of the invention comprise use of pH of at least about pH 1.022; this is the pH which Gasset et al. (1993) Proc. Natl. Acad. Sci. USA 90(1):1-5, determined to causes irreversible changes in the protein structure of abnormal prion, (PrP) 27-30; Gasset et al. found that the amount of beta sheet declined from 54.2% to 39.0%.

In one embodiment, methods of the invention comprise at least one step where hydroxide is in contact with the protein and the pH above 10.2; in one aspect the protein is in a fluid environment and under pressure and at temperature high enough to speed up the reaction. In one embodiment at method of the invention comprises at least one step above pH 10.8, which is the solubilization point of beef tissue. In one embodiment, a method of the invention comprises at least one step above pH 11.2, which is the point where optimum bone removal occurs with beef offal.

In alternative embodiments, in addition to alkali treatment as described herein (e.g., comprising at least one step having a stable pH above 10.2), methods of the invention can also comprise use of: formic acid; heat above 121° C.; pressure, e.g., above 3 atm; and other chemicals and/or enzymes which denature proteins and can hydrolyze terminal amino acids.

In alternative embodiments, methods of the invention comprise at least one step comprising use of a high pH under pressure of 160 to 240 psi on a 160° F. liquefied protein stream; this can reduce infectivity (e.g., of a prion) over 8 logs. In alternative embodiments, methods of the invention comprise at least one step comprising use of formic acid added under pressure to pH below 4.0; this can reduce infectivity over 6 logs. In alternative embodiments, methods of the invention comprise at least one step comprising use of a flash drying, e.g., on a drum; this should reduce infectivity over 3 logs at pH below 4.0.

Thus, the invention provides methods that can be an effective recycling process to destroy infectious agents while at the same time provide a method to recover valuable animal proteins for animal feeds.

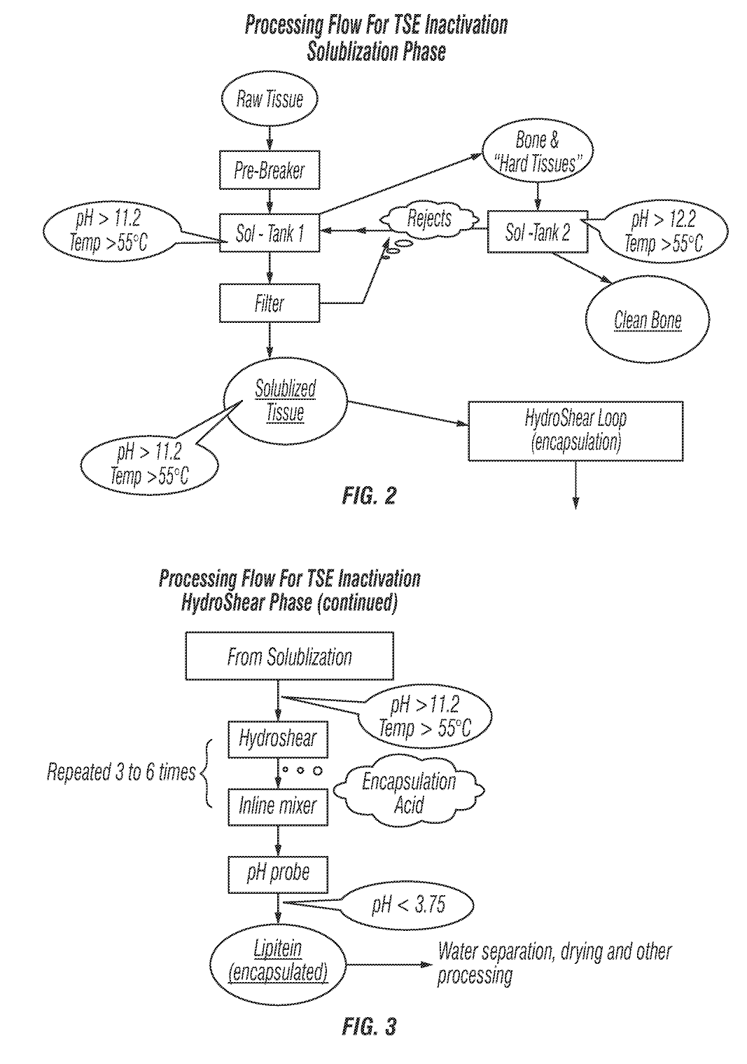

Exemplary processes of the invention comprise steps as set forth in FIG. 2 and FIG. 3, which illustrates an exemplary process of the invention comprising processing flow for TSE inactivation, including a “solubilization phase” (FIG. 2) and a hydroshear phase (FIG. 3). In summary, as illustrated in FIG. 2, raw tissue is processed in a “pre-breaker”, followed by a solubilization tank 1 at a pH of greater than about 11.2 and a temperature greater than about 55° C., followed by a filter, followed by harvesting of solubilized tissue at a pH of greater than about 11.2 and a temperature greater than about 55° C., followed by a hydroshear loop encapsulation of FIG. 3. As illustrated in FIG. 2, an optional path is an additional processing of some tissues, as appropriate, e.g., bone and “hard tissues”, in a second solubilization tank 2 at a pH of greater than about 12.2 and a temperature greater than about 55° C.; processed material from solubilization tank 2 is re-fed back to solubilization tank 1, and optionally clean bone is harvested from solubilization, tank 2. As illustrated in FIG. 3, the material from the exemplary process as illustrated in FIG. 2 is subjected to hydroshear followed by an inline mixer; and in alternative embodiments this can be repeated one, two, three, four, five, or six or more times; and “encapsulation acid” is added to a pH of about 3.75 or less (more acid), with the result being encapsulation of material, e.g., as illustrated, in FIG. 3, LIPITEIN® encapsulation. In alternative embodiments, the encapsulated material can be further processed, e.g., by water separation, drying and/or other processing steps, e.g., as described herein.

The invention will be further described with reference to the following examples; however, it is to be understood that the invention is not limited to such examples.

A number of embodiments of the invention have been described. Nevertheless, it will be understood that various modifications may be made without departing from the spirit and scope of the invention. Accordingly, other embodiments are within the scope of the claimed invention.

The following examples are offered to illustrate, but not to limit the claimed invention.

### EXAMPLES

### Example 1

### Exemplary Process of the Invention

The following Example describes exemplary methods of the invention used to further process LIPITEIN® (Parma Laboratories, Inc., Parma Id.), a processed fluid milk for calves. The LIPITEIN® currently produced from daily products is comprised of 11% to 13% total solids. The exemplary process of this invention described in this example removes or concentrates the liquid portion to greatly reduce the amount of moisture being shipped in the finished product; thus, the solids are dried economically, and the market for the product is significantly expanded beyond fluid milk for calves.

It was observed that dairy products with a pH of less than 5.8 following encapsulation can be separated into solids and liquid by filtering the material across stainless steel screens. The tests described in this example evaluate the use of membrane technology to concentrate the supernatant organic materials that remain after the milk protein and fat solids in a form of LIPITEIN® produced by an exemplary process of this invention have been removed by screening or centrifugation.

LIPITEIN® (produced from dairy products) was pre-processed using a screening system comprising 60 and 200 mesh SS screens under vacuum. Test material was titrated to pH 4.5 with phosphoric acid. The liquid recovered was then centrifuged. The recovered solids were also centrifuged. The solids dry matter was determined using an infrared drying test unit. The solids were 24% dry matter.

The liquid phase after centrifugation was observed; and it was determined that a membrane system could be used to recover additional dry matter, which would clean the fluid to the point it could be processed by a city sewer system. A UF polyvinylidene fluoride (PVDF)-30K membrane was used.

Additional tests were conducted adjusting the liquid to about pH 5.7. It appeared that between pH 5.7 and pH 4.5, the solids and liquid separated well. Four tubes were filled. The volume of the fluid and the solids was determined. Based on volume measures, the solids were 20.6% and the liquid was 79.4%. It is estimated that one-half to one percent of the dry matter of the test material was found in the liquid. Most of the dry matter of the liquid would be soluble carbohydrates, organic acids, nitrogenous, compounds (e.g., proteins) and ash.

To produce enough fluid for the membrane test, eighteen 400 ml centrifuge tubes were filled with the test material and were centrifuged at 2900 rpm for 10 minutes. The liquid was harvested for the membrane test, and the solids were spread on a drying pan and dried in a commercial drying system at 180° F. for approximately 40 minutes.

The liquid was placed in the feed tank of a membrane system, and the UF-PVDF-30k membrane was placed in the holder of the test equipment.

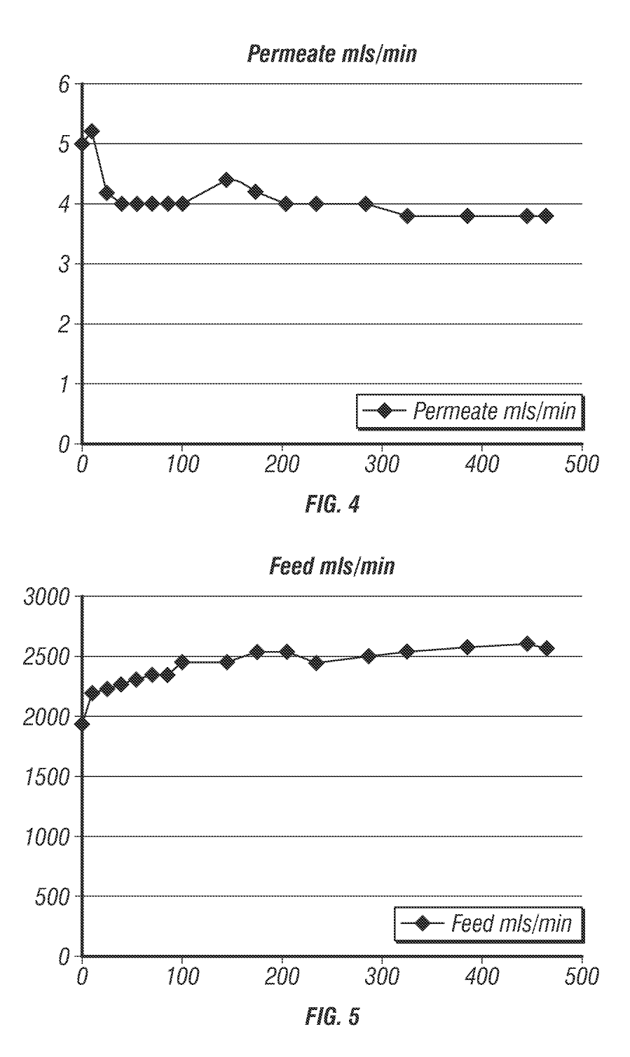

Data of operating pressures was recorded, as graphically illustrated in FIG. 4 (permeate mls/minute (min), and FIG. 5 (feed mls/min). This data confirms that membrane concentration of the fluid phase LIPITEIN® is practical.

The optical density of fluid post- and pre-filtering through UF-PVDF-30K membrane was measured. The fluid pre-filtering has a turbidity of 147, and post-filtering a turbidity of 1.67. A comparison of centrifuged test material at normal (physiologic, or about pH 7 to 7.4) pH for fluid feeding to calves and material at pH 5.6 was made; protein had precipitated/flocculated out of the pH 5.6 acidified material.

After determining that the optimal pH range is between 5.7 and 4.5, it was decided to test the product through a commercial centrifuge. The test material was adjusted to pH 5.25 and heated to 40° C. Centrifugation was started, and the temperature was then raised to 55° C. Good fluid removal was found at both temperatures. The centrifuge was set up with a standard cone set and operated on a 3.5 minute solids dump cycle. After centrifugation the solids were 22.5% of the volume, and the liquid was 77.5%. The turbidity of the commercial centrifuge liquid indicated additional centrifuging or filtering might be needed before sending the fluid to a membrane system.

An analysis of the centrifuge fluid is in Table 1, below; and a nutrient analysis of the dried solids from this process are shown below in Table 2.

Conclusions: Using an exemplary process of this invention, as described in this example, LIPITEIN® produced from recovered dairy products, and similar fluids, can be concentrated by lowering the pH to 5.7 or below. The product stream separates into a solid phase and a liquid phase when screened or centrifuged. The solids are 20-22% of the volume, and the liquid is 78-80%. The solids may contain as much as 99% of the protein and fat. The majority of protein and fat is recovered in the solids phase. The dried solids protein is 41.2%, fat 30.1%, ash 6.1%, calcium 0.4% and phosphorus 1.6%. This is a high value dried milk product.

This liquid, if prepared by practicing an exemplary process of this invention, which includes further filtering or centrifuging, can be further processed using membrane filters. The liquid harvested at the centrifuge had 712 ppm suspended solids (TSS), 25,800 ppm 5-Day BOD, 84,759 ppm COD, 1,550 ppm N, 403 ppm Oil and pH 4.0. The liquid harvested after the membrane had 56 ppm TSS and lipids were not detectable. Post membrane BOD was 21,700, COD was 44,688, N 207 and pH 4.4. The components concentrated by the membrane can be added to solids before drying.

In another test, using an exemplary process of this invention, as described in this example, LIPITEIN® was processed by pH adjustment followed by centrifugation for removal of the majority of solids. The liquid was then processed using ultra-filtration. The solids were dried using a commercial dryer.

As noted above, LIPITEIN® produced from recovered dairy products can be concentrated by lowering the pH to 5.7 or below. The product stream separates into a solid phase and a liquid phase when screened or centrifuged. The concentrated solids are 20-22% of the volume, and the liquid is 78-80%. The majority of protein and fat is recovered in the solids phase. The dried solids protein is 41.2%, fat 30.1%, ash 6.1%, calcium 0.4% and phosphorus 1.6%. This yielded a high value-dried milk product.

The liquid, if prepared by further filtering or centrifuging as described herein, can be processed using membrane filters. The liquid harvested at the centrifuge had 712 ppm suspended solids (TSS), 25,800) ppm 5-Day biological oxygen demand (BOD), 84,759 ppm chemical oxygen demand (COD), 1,550 ppm N, 403 ppm oil and pH 4.0. The liquid harvested after the membrane had 56 ppm TSS, and lipids were not detectable. Post membrane BOD was 21,700, COD was 44,688, N 207 and pH 4.4. The components concentrated by the membrane may be added to solids before drying.

We tested if simple enhancements could further improve the recovery of the valuable milk proteins and fats and improve the fluids for disposal (e.g., into a public waste (sewage) system). Improving recovery of anions, such as phosphate from the acid addition, would have a positive effect on disposal. Improved protein and fat recovery would have additional positive effects.

Two-batches of milk-based LIPITEIN® were produced in the laboratory using similar proportions of dairy products as used in the production of commercial LIPITEIN® based fluid milk product. Cottage cheese, yogurt and sour cream were mixed, warmed and treated with a calcium hydroxide suspension, and the pH raised to pH 11.5 following the steps out lined in U.S. Pat. No. 5,514,388. The premix had a smooth, creamy appearance. This premix was mixed with milk (50 gram premix to 1,350 gram milk), and the ending pH was 9.6. This mixture was subjected to high shear mixing as described in U.S. Pat. No. 5,514,388, as phosphoric acid was added until the pH was at 5.0. The ending pH of 5.0 was selected from previous results. The product was similar to commercial, milk-based LIPITEIN® and was ready for fluid and solids separation.

The laboratory test material used was a milk-based LIPITEIN® produced in a 1.5 liter quantity using sour cream, yogurt, cottage cheese, whole milk and 2% fat milk in composition similar to LIPITEIN® produced commercially. The pH of the laboratory test material was adjusted to pH 5.0 or lower using phosphoric acid in a blender. Sixty ml quantities were separated into liquid and solid phases following centrifuging for 20 minutes. Stage 1 was the first centrifuge step. The solids and liquids were separated. In Stage 2, the liquid was treated with a concentrated calcium hydroxide suspension to raise the pH above 7.0 and centrifuged a second time for 20 minutes.

Following stage 1 centrifuging, the liquid was poured from the solids.

Summary Data - Batch 2 separation
Stage 2 separated fluid phase composition

| | |
|---|---|
| Organic Matter | 64,103 mg/Kg |
| Phosphorus | 298 mg/Kg |
| Fatty Acids | 700 mg/Kg |
| Nitrogen | 1,760 mg/Kg |

Component recovery in solid phase

| | |
|---|---|
| Organic Matter | 65.7% |
| Phosphorus | 90.8% |
| Fatty Acids | 98.3% |
| Nitrogen | 79.1% |

Separation by volume

| | |
|---|---|
| Solid phase | 35% |
| Liquid phase | 65%. |

Separation of Batch 2 milk-based LIPITEIN® was of a similar magnitude as seen in previous tests.

Based on this information, it was decided to test the separation of the milk-based LIPITEIN® product from the commercial process using two different pH levels. The pHs selected were 5.8 pH and a pH below 3.5.

Commercial production milk-based LIPITEIN® samples, processed using the method described in U.S. Pat. No. 5,514,388, were shipped overnight to the testing facility. The pH of the product upon arrival was 6.15. Stage 1 processing was completed on two subsamples by lowering the pH's to 5.89 and 3.38.

Stage 2 processing was the same for both samples with ending pHs between 7.48 and 7.51. Stage 2 process comprised using Stage 1 recovered liquid and adjusting it up pH with Ca(OH)₂, i.e., stage 1 fluid phase liquids were adjusted up (made a more alkaline pH) with Ca(OH)₂ pH 7.48-7.51. For “Stage 2” recovered liquid, the liquid from stage 1 at pH 3.38 is clearer than liquid from stage 1 at pH 5.89. Analysis of fluid from Stage 2 processing is shown in Table 4, below. As summarized by the data in Table 4, the ash (mineral) of Stage 2 liquids is lower for the sample (41327) that was lowered to pH 3.38 in the Stage 1 process. This indicates better mineral recovery in the solids when the Stage 1 process is at the lower pH.

In summary, milk-based LIPITEIN® produced using the methods described in U.S. Pat. No. 5,514,388, can be separated into liquid and solids phases. The milk-based LIPITEIN® can be processed further by lowering the pH between 6.0 and 3.0 then using centrifugation or vacuum screening for separation into a liquid and solid phases. Following removal of the solids, the pH of the liquid phase is then elevated to 7.0 to 8.5, and this mixture is separated into liquid and solid phases using centrifugation or vacuum screening. The recovered solids from this step can be added to the solids from the first separation. The recovered liquid will be at a pH where it can be digested for further reductions in organic matter before normal waste water disposal.

TABLE 3
Batch 2 laboratory results & recovery calculations

| | | | | | Batch 2, | Batch 2, |
|---|---|---|---|---|---|---|
| | | | Batch 2, | Batch 2, | Second | Dried |
| | Batch 2, | Batch 2, | Milk | Second | Centrifuge | Second |
| Sample | pH 5.0 | Milk Solids | Liquid | Liquid | Solids | Solids |
| Dry Matter | 13.19% | 27.85% | 7.17% | 6.93% | 31.73% | 96.86% |
| Moisture | 86.81% | 72.15% | 92.83% | 93.07% | 68.27% | 3.14% |
| Crude Protein (Dry) | 25.93% | 36.55% | | 15.87% | | |
| Fatty Acids | 20.09% | 33.18% | | 1.01% | | |
| (Hydrolysis, Dry) | | | | | | |
| Ash (Dry) | 7.96% | 6.10% | 11.72% | 7.50% | | 43.94% |
| Calcium (Dry) | 1.52% | 1.58% | | 0.58% | | |
| Phosphorus (Dry) | 1.59% | 1.26% | | 0.43% | | |
| Organic Matter (Dry) | 92.04% | 93.90% | 88.28% | 92.50% | | 56.06% |
| Dry Matter ppm asis | | | | | | |
| Organic Matter ppm asis | 121,401 | | 63,297 | 64,103 | | |
| Phosphorus ppm asis | 2,097 | | | 298 | | |
| Fatty Acids ppm asis | 26,499 | | | 700 | | |
| Nitrogen ppm asis | 5,472 | | | 1,760 | | |
| Stage 1 recovery by | | 26.50% | 73.50% | Stage 1 Liquid | | |
| weight | | | | | | |
| Stage 2 recovery by ratios | | | | | | |
| Stage 2 solids OM asis | 56.06% | | 0.0422 | 11.6% | Stage 2 Solids | |
| Stage 1 liquid OM asis | | 88.28% | | | | |
| Stage 2 liquid OM asis | 92.50% | | 0.3222 | 88.4% | Stage 2 Liquid | |
| Phosphorus Recovery | 90.8% | | | | | |
| Organic Matter Recovery | 65.7% | | | | | |
| Fatty Acid Recovery | 98.3% | | | | | |
| Nitrogen Recovery | 79.1% | | | | | |
| Reduction in weight | 65% | | | | | |

TABLE 4
Analysis of fluid from Stage 2 processing.

| | | Alternative |
|---|---|---|
| | Alternative | Feeds, Milk |
| | Feeds, Milk | LIPITEIN ® |
| | LIPITEIN ® | Tubes 1&2, |
| | Tubes 3, 4, 5, 6 | Stage 2 |
| Sample | Stage 2 Liquid | Liquid |
| Dry Matter | 5.72% | 6.35% |
| Moisture | 94.28% | 93.65% |
| Crude Protein (Dry) | | |
| Fatty Acids | | |
| (Hydrolysis, Dry) | | |
| Ash (Dry) | 3.85% | 5.04% |
| Calcium (Dry) | | |
| Phosphorus (Dry) | | |
| Organic Matter(Dry) | 96.15% | 94.96% |
| Dry Matter ppm asis | | |
| Organic Matter ppm asis | 54,998 | 60,300 |
| Dry Matter | 5.72% | 6.35% |
| Moisture | 94.28% | 93.65% |
| Crude Protein (Dry) | | |
| Fatty Acids | | |
| (Hydrolysis, Dry) | | |
| Ash (Dry) | 3.85% | 5.04% |
| Calcium (Dry) | | |
| Phosphorus (Dry) | | |
| Organic Matter(Dry) | 96.15% | 94.96% |
| Dry Matter ppm asis | | |
| Organic Matter ppm asis | 54,998 | 60,300 |

### Example 2

### Exemplary Process of the Invention for Biofluids

The following Example describes exemplary methods of the invention that can be used to process biofluids such as dairy or soy milk (including fortified and/or flavored, e.g., chocolate milk), cream, fruit juice, liquid yogurt, liquefied cheese and the like.

Milk delivery trucks return various out of date or off specification fluid milk, chocolate milk, cream, soy milk, ice cream, various fruit juices and other dairy-based and/or soy-based liquid products in containers (e.g., retail containers) to a commercial processing facility. These materials are processed using the method and equipment described in U.S. Pat. No. 5,514,388, and the resulting product is known as LIPITEIN® (Parma Laboratories, Inc., Parma Id.), a stable, liquid that is transported with milk tankers to calf raisers: The calf raisers feed the LIPITEIN® as a replacement (substitution) for fluid milk and/or milk replacers (substitutions) to young calves. The LIPITEIN® is a fluid comprised of 9% to 13% solids at a pH between 6.0 and 6.8. This material contains milk fat and protein as well as some milk sugars and lactic acid salts from the fermentation of the liquid products before processing.

The liquid products are removed from the packaging by rupturing in mechanical crushers. The liquid product is pumped into a 6,000 gallon holding tank where sodium hydroxide or potassium hydroxide or calcium hydroxide or combinations of these chemicals are added to obtain a pH between about pH 9.5 and about pH 10.5, or obtain a pH between about pH 9.0 and about pH 11.5; or about pH 9, 9.5, 10, 10.5, 11, 11.5 or higher. The liquid product is heated above the melting point of the milk fat occurring in the liquid product (120° F. to 160° F.), thoroughly mixed, screened to remove small particles of packaging materials, then pumped past three HYDRASHEAR™ devices inline (e.g., as described in U.S. Pat. No. 5,575,561) and phosphoric acid is injected immediately behind two of the devices under pH control. The pH of the material is lowered to 6.3 to 6.1. The liquid LIPITEIN® is then cooled to 35° F. to 42° F. before shipment.

The invention provides methods for concentrating protein encapsulated in fat. It was observed that lowering the pH of the liquid LIPITEIN® between 5.8 and 3.3 caused the protein encapsulated fat to precipitate. It was demonstrated that the precipitate could be separated from the liquid by filtering under vacuum through a 200 mesh stainless screen. It was demonstrated that the precipitate could be separated from the liquid by centrifugation. It was demonstrated that lowering the pH to 4.5 or to pH 3.3 produced a much clearer fluid on separation.

Based on the above observations the pH of the liquid LIPITEIN® was adjusted to 5.25 and centrifuged at 40° C. and 55° C. using a commercial liquid/solids ejector centrifuge. The recovered solids were 22.5% of the volume and the liquid was 77.5%. The solids were dried on a commercial tray dryer. The recovered and dried solids contained nearly 99% of the protein and fat of the liquid LIPITEIN®. The dried solids were 41.2% protein, 30.1% fat, 6.1% ash, 0.4% calcium and 1.6% phosphorus.

The liquid recovered from the separation had turbidity greater than 1000. This liquid subjected to membrane filtration across a UF-PVDF-30K membrane and the turbidity of the filtered fluid dropped to 1.67. Post filtration fluid had 56 mg/L suspended solids, no detectable lipids, 21,700 mg/L BOD, 207 mg/L Nitrogen and a pH 4.4.

Lowering the pH of liquid LIPITEIN® between 5.8 and 3.3 allowed separation of high value protein encapsulated lipid from low value fluid stream.

### Example 3

### Exemplary Process of the Invention for Biofluids Supplemented with Lipids

The following Example describes exemplary methods of the invention that can be used to process biofluids such as dairy or soy milk (including fortified and/or flavored, e.g., chocolate milk), cream, fruit juice, yogurt, liquid yogurt, cheese, liquefied cheese and the like, are also processed by addition of fats and/or lipids.

The liquid or semiliquid products are removed from the packaging by rupturing in mechanical crushers. Five thousand gallons of liquid product is pumped intro a 6,000 gallon holding tank where sodium hydroxide or potassium hydroxide or calcium hydroxide or combinations of these chemicals are added to obtain a pH between 10.5 and 11.0. The liquid product is heated above the melting point of the milk fat occurring in the liquid product (120° F. to 160° F.) and screened to remove small particles of packaging materials, eighty (80) gallons of animal tallow, preheated to above the melting point (120° F. to 160° F.) is gradually injected via a gear pump into the pH 11.0 to 11.5, liquid product and thoroughly mixed. The liquid product and animal tallow is then pumped past three HYDRASHEAR™ devices inline (e.g., as described in U.S. Pat. No. 5,575,561) and phosphoric acid is injected immediately behind two of the devices under pH control. The pH of the material is lowered to (was adjusted to) 3.8 and centrifuged using a commercial centrifuge, thereby recovering a solid.

The recovered solids were less than 25% of the volume. The solids were dried on a commercial fray dryer. The recovered and dried solids contained nearly 99% of the protein and fat of the liquid LIPITEIN®. The dried solids were 41.2% protein, 42.1% fat, 5.7% ash, 0.4% calcium and 1.5% phosphorus. No observable free lipid was found on the dried material showing the added fat was encapsulated by the milk proteins. Lowering the pH of the final liquid between 5.8 and 3.3 allowed separation of high value protein encapsulated lipid from low value fluid stream.

### Example 5

### Exemplary Process of the Invention for Dairy Products

The following Example describes exemplary methods of the invention that can be used to process biofluids such as dairy or soy milk (including fortified and/or flavored, e.g., chocolate milk), cream, fruit juice, yogurt, liquid yogurt, cheese, liquefied cheese and the like, are also processed by addition of fats and/or lipids.

Approximately 2,500 gallons of out of date or off specification, solid milk-based products composed of one third yogurt, one third sour cream, and one third cottage cheese in the retail packaging was ruptured in mechanical crushers. Approximately 2,500 gallon of heated (90° F. to 130° F.) liquid products recovered as described in Example 2, above, with the pH adjusted to pH 11.0 to 11.5 is sprayed over the crushed contents and packaging of the solids (yogurt, sour cream and cottage cheese) liquefying the solids and removing the same from the packaging. This produces a combined liquid products and solid products stream at pH 11.0 to 11.5 and at 90° F. to 130° F.

Approximately 2,000 gallons of the combined liquid product and solid products stream is mixed with approximately 4,000 gallons of liquid product recovered (as described in Example 2, above, but without the addition of the alkalis) and mixed in a holding tank. The pH is measured and if the combined products are below pH 9.5 then additional sodium hydroxide or potassium hydroxide or calcium hydroxide or combinations of these chemicals are added to obtain a pH between 9.5 and 10.5. The combined product is heated above the melting point of the milk fat (120° F. to 160° F.), thoroughly mixed, screened to remove small particles of packaging materials, then pumped past three HYDRASHEAR™ devices inline (e.g., as described in U.S. Pat. No. 5,575,561) and phosphoric acid is injected immediately behind two of the devices under pH control. The pH of the material was adjusted to 3.8 and centrifuged using a commercial centrifuge. The recovered solids were between 25 and 35% of the volume. The solids were dried on a commercial tray dryer. The recovered and dried solids contained nearly 99% of the protein and fat of the liquid LIPITEIN®. The dried solids were 41.2% protein, 32.1% fat, 6.4% ash, 0.5% calcium and 1.3% phosphorus. No observable free lipid was found on the dried material showing the added fat was encapsulated by the milk proteins. Lowering the pH of liquid LIPITEIN® between 5.8 and 3.3 allowed separation of high value protein encapsulated lipid from low value fluid stream.

### Example 6

### Solubilization and Encapsulation of Proteins by Manipulating pH

The following Example illustrates exemplary titration curves that can be used for determining optimum acid and alkaline pH for solubilizing and encapsulating protein, respectively, for any initial composition used to practice this invention; for example, the methods of the invention can be used to process any biomass, including animal, plant, microorganism and/or algae biomass. In this example, optimum acid and alkaline pH for solubilizing and encapsulating was determined for fluid skim milk, fluid regular milk, whey protein and soy protein; the illustrated titration curve graphs are shown below.

In alternative embodiments, methods of the invention comprise adding a liquid comprising an acid (at the same time or any time during the process of the mixing, or shearing or sonicating) to: (i) lower the pH (make the liquid more acid) to between about pH 3 and pH 6, or about pH 3, about pH 4, about pH 5, or about pH 6, thereby generating a precipitate or flocculant comprising substantially most of the protein, lipid or lipid-encapsulated structures or protein-encapsulated structures out of the liquid, or (ii) lower the pH (make the liquid more acid) to the point where substantially most of the protein, lipid, or lipid-encapsulated structures or protein-encapsulated structures precipitate or flocculate out of the liquid. The optimum pH for any particular biomass or organic matter source can be determined, e.g., using the titration protocol described in U.S. Pat. No. 5,514,388. In particular, optimum pH of solubilization of protein can be determined as an alkali hydrogen ion difference on a graph in which rate of change in hydrogen ion concentration varies with acid equivalent units, e.g., as described in U.S. Pat. No. 5,514,388.

As described in Example 3, of in U.S. Pat. No. 5,514,388, in one embodiment, the titration protocol is determined by graphing data in a format where the rate of change in hydrogen ion concentration is plotted against the pH at equal units of acid or base addition. The optimum pH is determined from the point where the slope of the rate of change in hydrogen ion concentration versus uniform units of acid or base addition is essentially zero.

In one aspect, to determine the solubilization graph data points, a standard base is prepared by diluting 10 cc of a 40% sodium hydroxide solution to 2 liters with distilled water. A standard acid can be prepared by diluting 10 cc of reagent grade phosphoric acid to 2 liters with distilled water to determine encapsulation graph data points. The base equivalent unit for the acid and the acid equivalent unit for the base can be determined by titration of 50.5 ml of the standard base with 33.0 ml of standard acid to a pH of 7.02.

In one aspect, solubilization graph data points are determined by mixing/blending organic matter (biomass) and distilled water into a mixer or blender, e.g., a WARING™ blender, and titrating the mixture with additions of standard base (e.g., a 5.05 ml base addition was used in Example 3, of in U.S. Pat. No. 5,514,388). The stabilized pH measures of the titrated mixture are recorded for each uniform base addition. At each data point the rate of change in hydrogen ion concentration is calculated and recorded. Data points are graphed where they cover ah optimum solubilization point.

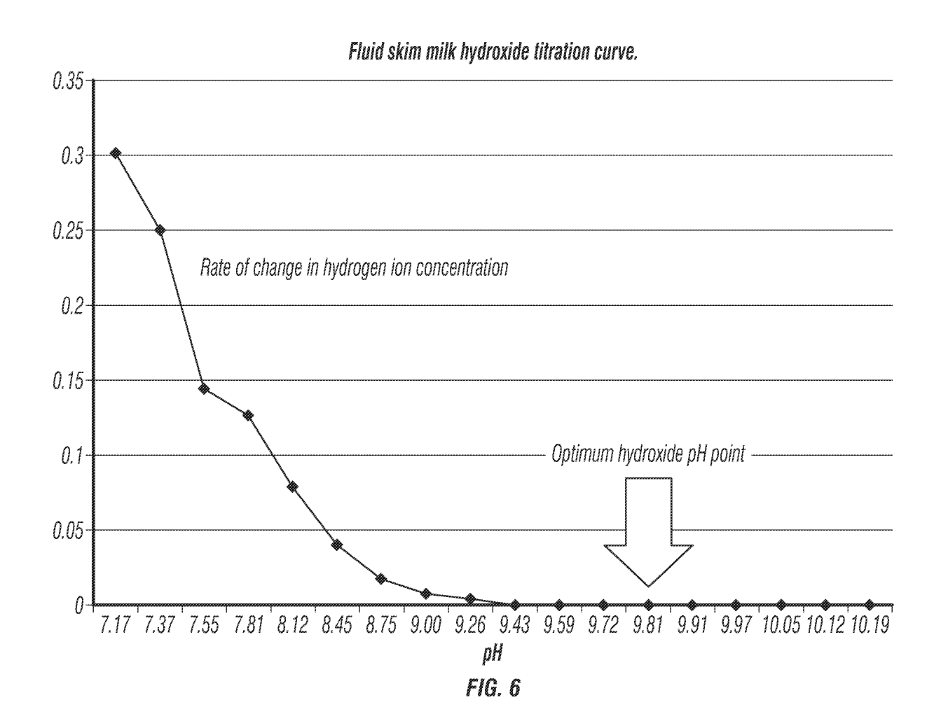

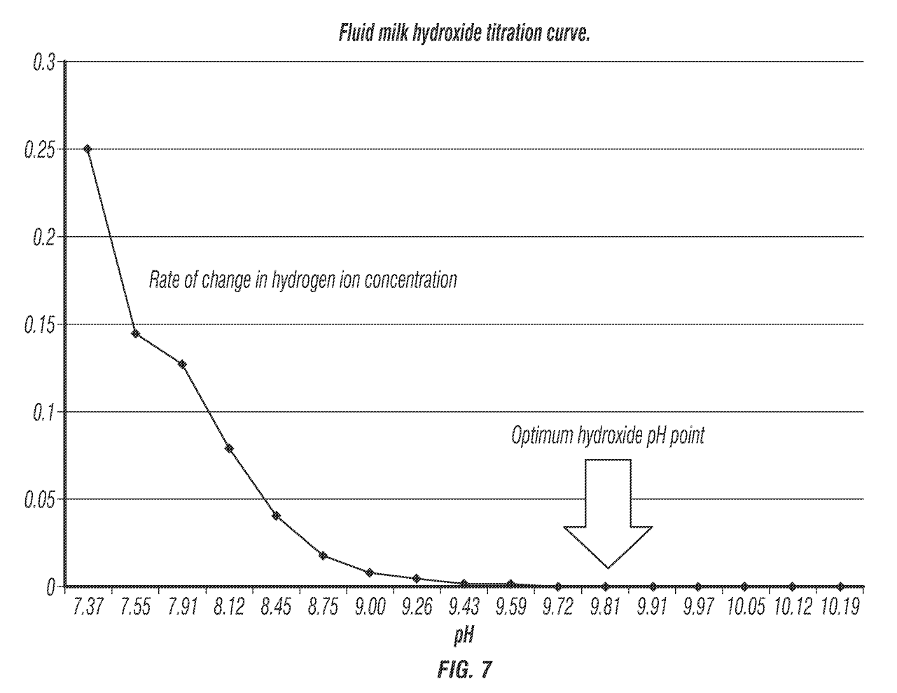

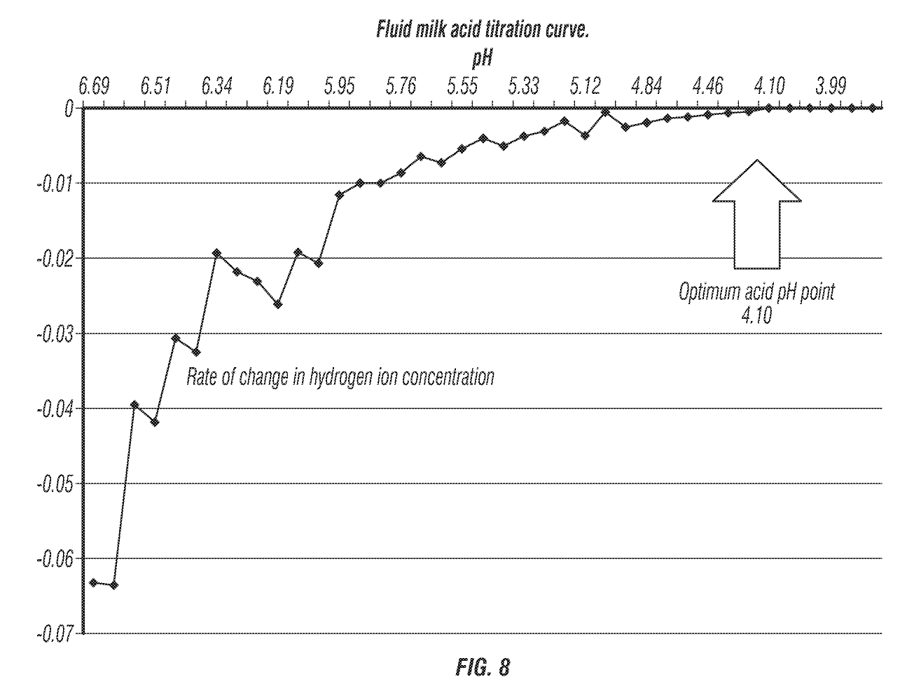

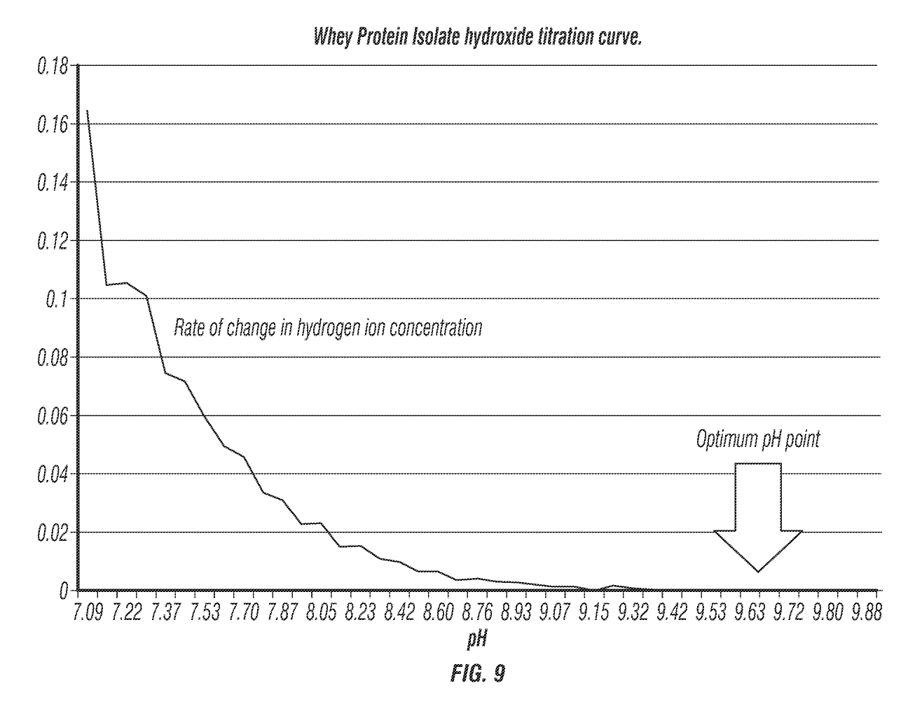

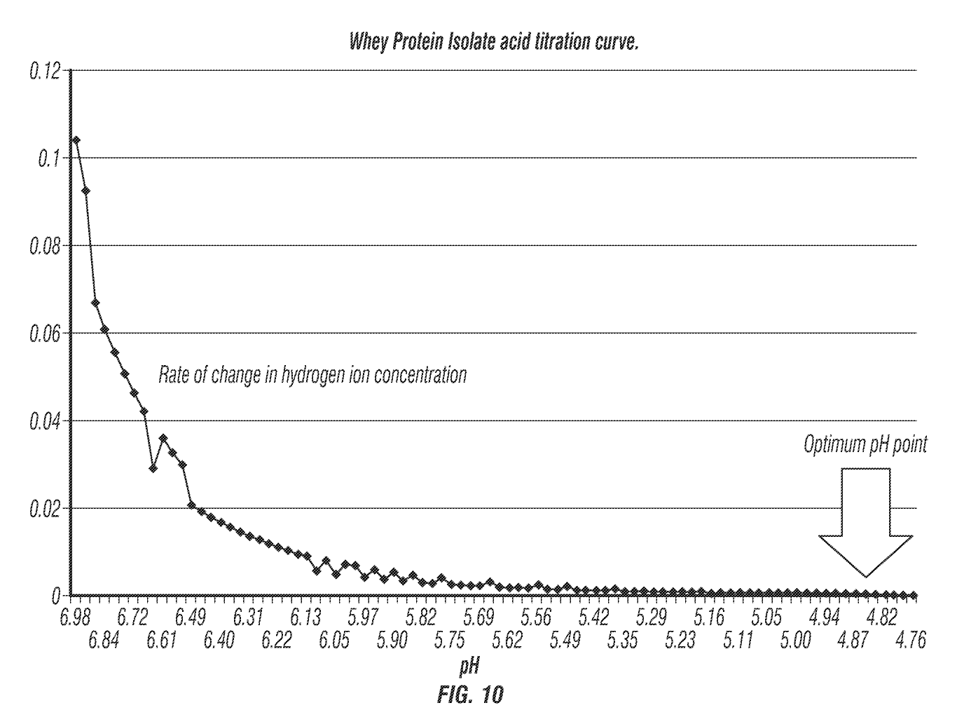

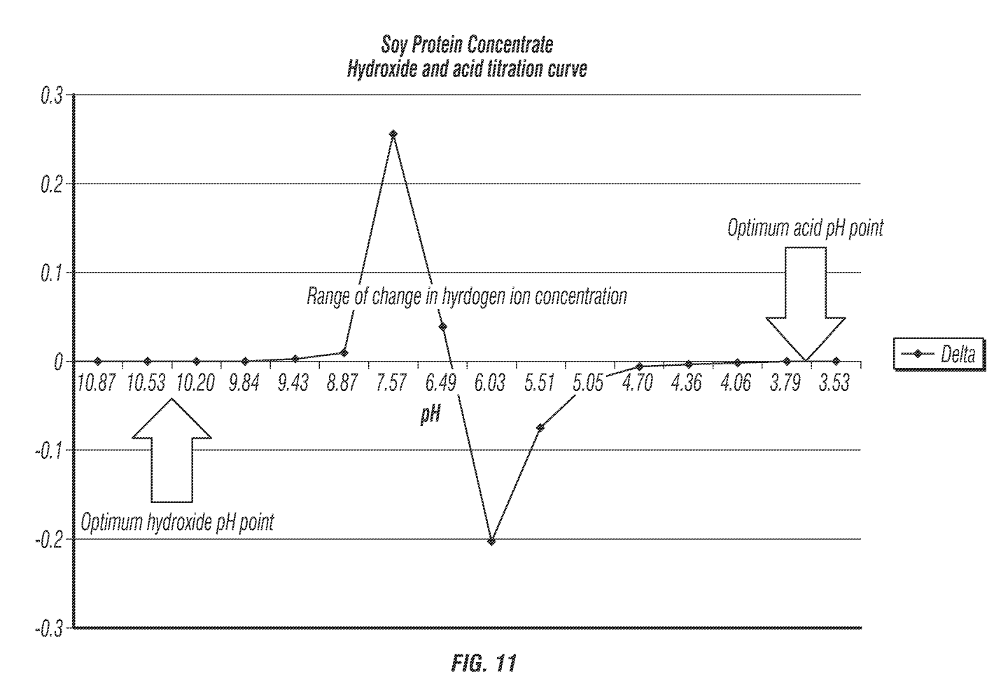

In one aspect, encapsulation graph data points are determined by mixing/blending organic matter (biomass) and distilled water into a mixer or blender, e.g., a WARING™ blender, and adjusting, the pH to a stabilized pH 10.3 with standard base then titrating the mixture with 5.0 ml additions of standard acid. The stabilized pH measures of the titrated mixture are recorded for each uniform 5.0 ml acid addition. At each data point the rate of change in hydrogen ion concentration is calculated and recorded. The data points between are graphed as they cover an optimum encapsulation point. Exemplary encapsulation titration curve graph data points are illustrated in FIG. 6 (showing an encapsulation titration curve graph for fluid skim milk), FIG. 7 (showing a hydroxide titration curve graph for fluid skim milk), FIG. 8 (showing an acid titration curve graph for fluid skim milk), FIG. 9 (showing a whey protein isolate hydroxide titration curve graph), FIG. 10 (showing, a whey protein isolate acid titration curve graph), and FIG. 11 (showing a soy protein concentrate hydroxide and acid titration curve graph).

In summary, in alternative embodiments, processes for protein encapsulation of lipid comprise treating a proteinaceous mixture with an alkali to raise the pH to where the proteinaceous mixture is solubilized and will form a gel, e.g., as described in U.S. Pat. No. 5,514,388. In one embodiment, the protein is solubilized without addition of extraneous water to the process. In alternative embodiments, the optimum pH of solubilization and the optimum pH of encapsulation are determined by titration for each proteinaceous mixture to be used as they vary depending on the types of proteins, as discussed above. Optimum pH of solubilization can be determined as an alkali hydrogen ion difference on a graph in which rate of change in hydrogen ion concentration varies with acid equivalent units. Rate of change of hydrogen ion difference is measured and when the slope of the titration curve is essentially zero, then addition of alkali is ceased. Optimum pH of encapsulation can be determined as an acid hydrogen ion difference on a graph in which rate of change in hydrogen ion concentration varies with acid equivalent units. Rate of change of hydrogen ion difference is measured and when the slope of the titration curve is essentially zero, then addition of acid is ceased. Depending on the particular starting, organic material and the desired end product, capsules can be created in a range at or below the pH of encapsulation, but using less than the amount of acid which would partially or completely break down the primary structure of the protein.

### Example 7

### Exemplary Formulation of the Invention for Human or Animal Consumption

The following Example illustrates an exemplary formulation of the invention for making e.g., a food, food supplement, nutritional supplement or dietary aid for human or animal consumption. This exemplary food, food supplement, nutritional supplement or dietary aid of the invention comprises soy protein encapsulating flax oil. However, the invention also provides variations of this exemplary method, e.g., using any plant or animal protein encapsulating any oil, e.g., a fish- or algae-derived oil.

This final product can be packaged as an oral formulation, e.g., as capsules, tablets, “soft-gels” and/or a liquid for oral consumption, and the like. In alternative embodiments, the final product can be used as a food additive or supplement, a nutritional aid or a dietary aid, and can be added to a food or feed in a manufacturing or packaging process.

### EXEMPLARY METHOD OF THE INVENTION

BATCH SHEET FOR OMEGA 3
LIPITEIN ®

| | | |
|---|---|---|
| Materials | | |
| Flax Oil | 8.9 kg | |
| Soy Protein Isolate (screened) | 7 kg | |
| Water (140 to 160° F.) | 37.8 kg | |
| Vitamin E 40% | 0.1 kg | 13 gms |
| | 53.8 kg | 9 gms |

| | |
|---|---|
| Slaked calcium oxide (15% w/w in water) | as needed to pH 10.3 to 10.6 |
| Phosphoric acid (25% P) | as needed to pH 3.8 to 3.5 |

Manufacturing (LIPITEIN ® -- Omega 3)
Soy protein isolate is screened dry to remove foreign materials.
Calcium oxide is mixed with water at 15% w/w and allowed to slake.
Flax oil is weighed into pail.
Vitamin E 40% is weighed on gram (gm) scale and added to pre-weighed
flax oil and mixed.
Soy protein isolate is weighed into pail.
Hot water between 140 and 160° F. is added to the preheated mix tank.
Bypass valves are set to allow circulation without passing through the
HYDRASHEAR ™.
Progressive pump is turned on to start circulation of the hot water.
Soy protein isolate is slowly added to tank and allowed to mix.
Slaked calcium oxide is slowly added until pH stabilizes between
10.3-10.6.
Flax oil/Vitamin E premix is slowly added and allowed to mix.
pH is checked and adjusted to 10.3-10.6, if needed.
Valves are switched allowing mixture to pass through the
HYDRASHEAR ™.
Mixture is sheared until smooth, mixed and no oil visible.
Acid injection pump is turned on.
The discharge valves are set and the Lipitein ® is transferred to
plastic pails.
Concentration (Lipitein ® -- Omega 3)
Vacuum screen tray is fitted with a 20-by-20-inch 200 mesh stainless steel
screen.
Wet/dry vacuum pump is connected to the vacuum screen tray and
turned on.
Lipitein ® is transferred to the vacuum screen and spread out at
½ to ¾ inch in depth.
Drying (Lipitein ® -- Omega 3)
The trays are placed in a forced air oven and dried at 95° to 105° C.
until dry.

| Analysis | After | Before |
|---|---|---|
| (Lipitein ® -- Omega 3) | Drying | Concentration |
| Dry Matter | 94.00% | 29.36% |
| Protein | 37.53% | 11.72% |
| Lipid | 52.77% | 16.48% |
| Ash | 3.52% | 1.10% |
| Calcium | 0.26% | 0.08% |
| Phosphorus | 0.22% | 0.07% |

### Example 8

### Exemplary Formulation of the Invention for Human or Animal Consumption

The following Example illustrates two exemplary formulations of the invention for making e.g., a food, food supplement, nutritional supplement or dietary aid for human or animal consumption. These two exemplary compositions, e.g., whether finally processed as a food, food supplement, nutritional supplement or dietary aid and the like, comprise skim milk and an encapsulating fat or oil, e.g., in one embodiment, a flax oil. However, the invention also provides variations of this exemplary method, e.g. using any plant or animal protein encapsulating any oil, e.g., a fish- or algae-derived oil.

These exemplary formulations comprise use of: a vitamin B supplement—Vitamin B₃, also called niacin or niacinamide, in the form of an inositil niacinate; a calcium supplement in the form of calcium oxide; and, a phosphate supplement in the form of H₃PO₄.

In alternative embodiments, alternative milk formulations are substituted for skim milk, or used with the skim milk, e.g., whole milk (typically about 3.25% milk fat), and/or 1% or 2% (fat content) milk.

Both of the following exemplary formulas of the invention can yield a fluid milk product suitable for drinking and/or eating, or using in a food or feed product for any reason. In one embodiment, the pH is lowered to approximately 4.4, at this acidic pH the concentrated product (e.g., fat or oil encapsulated by protein, e.g., flax oil encapsulated by milk protein) will separate. The concentrate can be a very smooth creamy yogurt like material suitable for yogurt replacements, or other similar food or feed uses. The liquid can be treated as described herein, using any alternative exemplary method of this invention.

In one embodiment, the concentrate is lipid-encapsulated with protein, lipid on the inside. The mixture is treated with base, well sheared as acid is added until the “encapsulation pH” is reached. If fluid milk product is desired then the process is stopped at this point. If the concentrated form of the product is desired then addition acid is added to about 4.4. This applies to both formulas, below. These two exemplary formulas comprise two (2) base formulas 1 with niacin and 1 without niacin. The two base formulas as described are fluid with and ending pH of 6.8 to 6.3. In one embodiment, to produce the concentrated version of the two formulas, additional acid is added to lower the pH to about 4.4. The lipid encapsulated by the protein is concentrated, and it is possible to remove a large amount of the moisture with simple settling and pouring off the excess liquid and collecting the concentrate.

| | | | | | Inositol | | | Vitamin E |
|---|---|---|---|---|---|---|---|---|
| | | | SkimMilk | Flaxoil | Niacinate | Ca(OH)2 | H3PO4 | Oil 20 |
| | Target | Formula | 95.859% | 2.154% | 0.581% | 0.854% | 0.552% | IU/drop |
| Batch Units | 1000 | | 958.6 | 21.5 | 5.8 | 8.5 | 5.5 | 18 drops for mix |
| Protein (gram) | 8.75 | 8.75 | | | | | | |
| Omega 3 (mg) | 3,150 | 3,150 | | | | | | |
| Insositol Niacinate | 1,575 | 1,575 | | | | | | |
| (mg) | | | | | | | | |
| Calcium (mg) | 1,575 | 1,575 | | | | | | |
| Phosphorus (mg) | 730 | 730 | | | | | | |
| Protein to Fat | | 1.45 | | | | | | |
| $/lb | $0.1665 | MIX | | | | | | |
| Mix Size (grams) | | 270.86 | 259.64 | 5.83 | 1.58 | 2.31 | 1.50 | |
| Water | 87.08% | 235.88 | 235.86 | 0.00 | 0.00 | 0.00 | 0.01 | |
| Dry Matter | 12.92% | 34.98 | 23.78 | 5.83 | 1.58 | 2.31 | 1.48 | |
| Protein | 3.23% | 8.75 | 8.75 | 0.00 | 0.00 | 0.00 | 0.00 | |
| Fat | 2.23% | 6.04 | 0.21 | 5.83 | 0.00 | 0.00 | 0.00 | |
| Ash | 0.72% | 1.95 | 1.95 | 0.00 | 0.00 | 0.00 | 0.00 | |
| CHO | 4.75% | 12.88 | 12.88 | 0.00 | 0.00 | 0.00 | 0.00 | |
| Calcium | 0.58% | 1.58 | 0.32 | 0.00 | 0.00 | 1.25 | 0.00 | |
| Phosphorus | 0.27% | 0.73 | 0.26 | 0.00 | 0.00 | 0.00 | 0.47 | |
| Potassium | 0.15% | 0.41 | 0.41 | 0.00 | 0.00 | 0.00 | 0.00 | |
| Sodium | 0.04% | 0.11 | 0.11 | 0.00 | 0.00 | 0.00 | 0.00 | |
| Magnesium | 0.01% | 0.03 | 0.03 | 0.00 | 0.00 | 0.00 | 0.00 | |
| Omega-3 | 1.16% | 3150.0 | 0.00 | 3150.00 | 0.00 | 0.00 | 0.00 | |
| InositolNiacinate | 0.58% | 1575.0 | 0.00 | 0.00 | 1575.00 | 0.00 | 0.00 | |
| Protein to Fat | | 1.45 | | | | | | |

| | | | | | Inositol | | | Vitamin E |
|---|---|---|---|---|---|---|---|---|
| | | | SkimMilk | Flaxoil | Niacinate | CaO | H3PO4 | Oil 20 |
| | Target | Formula | 96.621% | 2.171% | 0.000% | 0.651% | 0.557% | IU/drop |
| Batch Units | 1000 | | 966.2 | 21.7 | 0.0 | 6.5 | 5.6 | 19 drops for mix |
| Protein (gram) | 8.75 | 8.75 | | | | | | |
| Omega 3 (mg) | 3,150 | 3,150 | | | | | | |
| Insositol Niiacinate (mg) | 0 | 0 | | | | | | |
| Calcium (mg) | 1,575 | 1,575 | | | | | | |
| Phosphorus (mg) | 730 | 730 | | | | | | |
| $/lb | $0.1385 | MIX | | | | | | |
| Mix Size (grams) | | 268.72 | 259.64 | 5.83 | 0.00 | 1.75 | 1.50 | |
| Water | 87.78% | 235.88 | 235.86 | 0.00 | 0.00 | 0.00 | 0.01 | |
| Dry Matter | 12.22% | 32.85 | 23.78 | 5.83 | 0.00 | 1.75 | 1.48 | |
| Protein | 3.26% | 8.75 | 8.75 | 0.00 | 0.00 | 0.00 | 0.00 | |
| Fat | 2.25% | 6.04 | 0.21 | 5.83 | 0.00 | 0.00 | 0.00 | |
| Ash | 0.72% | 1.95 | 1.95 | 0.00 | 0.00 | 0.00 | 0.00 | |
| CHO | 4.79% | 12.88 | 12.88 | 0.00 | 0.00 | 0.00 | 0.00 | |
| Calcium | 0.59% | 1.57 | 0.32 | 0.00 | 0.00 | 1.25 | 0.00 | |
| Phosphorus | 0.27% | 0.73 | 0.26 | 0.00 | 0.00 | 0.00 | 0.47 | |
| Potassium | 0.15% | 0.41 | 0.41 | 0.00 | 0.00 | 0.00 | 0.00 | |
| Sodium | 0.04% | 0.11 | 0.11 | 0.00 | 0.00 | 0.00 | 0.00 | |
| Magnesium | 0.01% | 0.03 | 0.03 | 0.00 | 0.00 | 0.00 | 0.00 | |
| Omega-3 | 1.17% | 3150.0 | 0.00 | 3150.00 | 0.00 | 0.00 | 0.00 | |
| InositolNiacinate | 0.00% | 0.0 | 0.00 | 0.00 | 0.00 | 0.00 | 0.00 | |
| Protein to Fat | | 1.45 | | | | | | |

A number of aspects of the invention have been described. Nevertheless, it will be understood that various modifications may be made without departing from the spirit and scope of the invention. Accordingly, other aspects are within the scope of the following claims.

1. A method for processing and recovering organic matter from a biofluid, biosemisolid or biosolid (biomass) comprising a protein and a lipid, comprising:
(a) providing a first liquid and/or semisolid or paste comprising an organic matter (a biomass) in a liquid, a semisolid or paste, a colloidal and/or a solid or a particulate form;
(b) adding to the first liquid and/or semisolid or paste of step (a) a second liquid comprising a base to raise the pH (make the liquid and/or semisolid or paste more alkaline) of the first and second liquid mixture to:
(i) between about pH 8 and about pH 13, or between about pH 11 and about pH 12, or at least about pH 8, about pH 9, about pH 10, about pH 11, about pH 12 or about pH 13,
(ii) a point at which the protein flocculates or coagulates, or the protein and lipid form an emulsion, or
(iii) a point at which the protein and lipid form a lipid-encapsulated structure (a liposome or vesicle comprising the lipid encapsulating the protein) or a protein-encapsulated structure (a liposome or vesicle comprising the protein encapsulating the lipid),
and raising the temperature of the first liquid and/or semisolid or paste to a point at or above the melting point of the lipid, wherein the temperature is raised either before, during and/or after adding the second liquid to the first liquid;
(c) mixing, and shearing or sonicating the liquid of step (b), and at the same time or any time during the process of the mixing, or shearing or sonicating, adding a liquid comprising an acid to:
(i) lower the pH (make the liquid more acid) to between about pH 3 and pH 6, or about pH 3 or less, about pH 4, about pH 5, or about pH 6, or about pH 3.0 or less, thereby generating a precipitate or flocculant comprising substantially most of the protein, lipid or lipid-encapsulated structures or protein-encapsulated structures out of the liquid, or
(ii) lower the pH (make the liquid more acid) to the point where substantially most of the protein, lipid, or lipid-encapsulated structures or protein-encapsulated structures precipitate or flocculate out of the liquid; and
(d) separating or removing some, all or substantially most of the liquid from the precipitate or flocculate, or separating or removing some, all or substantially most of the precipitate or flocculate from the liquid.

2. The method of claim 1, wherein
(a) the first liquid and/or semisolid or paste comprising an organic matter (a biomass) comprises a mixture of carbohydrates, proteins and lipids; or
(b) the colloidal form comprises a colloidal aerosol, a colloidal emulsion, a colloidal foam, a colloidal dispersion or a hydrosol.

3. The method of claim 1, wherein the first liquid and/or semisolid or paste comprising an organic matter (a biomass) comprises an animal-based or an animal-derived organic matter or a plant-based or a plant-derived organic matter or a microorganism-based or a microorganism-derived organic matter or an algae-based or an algae-derived organic matter, or any combination thereof.

4. The method of claim 3, wherein the animal-based or animal-derived liquid organic matter comprises one or more dairy or meat products dairy-derived or meat-derived products, or one or more fish or fish-derived products.

5. The method of claim 4, wherein the one or more dairy products comprises milk, cheese, cream cheese, cottage cheese, yogurt or sour cream, or any combination thereof.

6. The method of claim 5, wherein the one or more dairy products comprises LIPITEIN®.

7. The method of claim 3, wherein the protein-encapsulated structure (liposome or vesicle comprising protein encapsulating lipid) comprises:
(a) a plant-derived or an animal-derived protein encapsulating an animal-, plant- or fish-derived oil;
(b) a soy-derived or dairy-derived protein encapsulating an animal-, plant- or fish-derived oil;
(c) the method of (a) or (b), wherein the dairy-derived protein comprises a whey (milk plasma) or equivalent; or the plant-derived oil comprises a flax oil or linseed oil (flax seed oil).

8. The method of claim 3, wherein the plant-based or plant-derived first liquid and/or semisolid or paste organic matter (biomass), or the algae-based or algae-derived liquid and/or semisolid or paste organic matter (biomass), comprises
(a) a rice, flaxseed (linseed), rapeseed (canola), sunflower, cottonseed, peanut, wheat, soy, barley, sorghum, oats, potato or corn plant, product or extract, or any combination thereof;
(b) a plant, product or extract from a species from a genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Phormium, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, or Zea, or any combination thereof;
(c) a plant, product or extract from a cotton, a silk cotton tree (Kapok, Ceiba pentandra), desert willow, a creosote bush, a winterfat, a balsa, a ramie, a kenaf, a hemp, a roselle, a jute, a sisal abaca, a flax, a member of the Linaceae family, a Linum usitatissimum, a Phormium tenax or a Phormium cookianum or any combination thereof;
(d) a composition, product or extract comprising or derived from a macroalgae, a seaweed; a red seaweed; a specie of the genus Ulva, Chlorophyta, Charophyta, Rhodophyta, Phaeophyceae, Porphyra, Gracilaria, Grateloupia, Kappaphycus or Ceramium; a green seaweed; a brown seaweed or brown algae; a kelp; a specie of the genus Laminaria; or, a Laminaria japonica;
(e) a plant, composition, product or extract comprising or derived from a halophyte, a plant or specie of the genus Salicornia or Spartina, or a Spartina alterniflora (smooth cordgrass or saltmarsh cordgrass) or a Spartina patens (saltmeadow cordgrass); or
(f) a composition, product or extract comprising or derived from a microalgae (also called phytoplankton, microphytes, planktonic algae).

9. The method of claim 3, wherein the plant-based or plant-derived first liquid and/or semisolid or paste organic matter (biomass) comprises a plant, food, fish, fowl or poultry, or animal waste stream; or a plant, food, fowl or poultry, fish or animal processing waste stream, or a human, fowl or poultry, fish or animal excrement (sewage) processing plant waste stream, or any combination thereof.

10. The method of claim 3, wherein the first liquid and/or semisolid or paste organic matter (biomass) comprising the plant-based or plant-derived liquid organic matter is manufactured by a process comprising: (a) raising the pH to at least pH 10.2; (b) raising the pH to between about pH 10.2 and pH 11.5; or, (c) the method of (a) or (b), and wherein at least one step of the process comprises raising the temperature to between about 140° C. and 180° C.

11. The method of claim 1, wherein in step (b) the pH is raised (make the liquid more alkaline) to about pH 11.0, pH 11.1, pH 11.2, pH 11.3, pH 11.4, pH 11.5 or pH 11.6.

12. The method of claim 1, wherein the base added in step (b) comprises a calcium hydroxide, or a Ca(OH)₂ (or slaked lime or hydrated lime), a sodium hydroxide (lye or caustic soda), a potassium hydroxide, a calcium oxide, a magnesium hydroxide, or an alkaline solution (an alkali), or any combination thereof.

13. The method of claim 1, further comprising after step (b) adding to the raised pH liquid a third liquid comprising an organic matter and a buffer in an amount sufficient to lower the pH (make the liquid more acid) in any amount that lowers the pH (makes the liquid more acidic) to an approximately neutral pH (or about pH 7).

14. The method of claim 13, wherein the third liquid comprises a milk, or a milk-based or milk-derived liquid.

15. The method of claim 1, wherein the mixing or shearing of step (c) comprises a high shear mixing or a high-shear homogenization in a mixing or a shearing device.

16. The method of claim 15, wherein the mixing or shearing comprises:
(a) pumping the liquid of step (b) to the mixing or shearing device with a line pressure above about 25 psi., or in the range of 60 to 150 psi;
(b) using a HYDRASHEAR™ shearing device;
(c) using an in-line injector immediately following the mixing or shearing to add the liquid comprising an acid; or
(d) the method of 16(c), wherein sufficient acid is added to lower the pH (acidify) to a pH of protein-lipid encapsulation, or a lower (more acid) pH.

17. The method of claim 1, wherein the liquid comprising an acid added in the mixing of step (c) comprises a phosphoric acid, a nitric acid, a boric acid, a hydrochloric acid, a sulfuric acid, a lactic acid, an acetic acid, a propionic acid (propanoic acid), a formic acid, a citric acid, an oxalic acid, or an organic acid, or any combination thereof.

18-36. (canceled)

37. A method for processing and recovering organic matter from a dairy-based, soy-based and/or a fruit-based liquid, comprising:
(a) providing a dairy-based, soy-based and/or a fruit-based first liquid, colloid and/or semisolid or paste, wherein the dairy-based and/or a fruit-based first liquid, colloid and/or semisolid or paste comprise a protein and a lipid;
(b) adding to the dairy-based and/or a fruit-based first liquid, colloid and/or semisolid or paste of step (a) a second liquid comprising a base comprising a sodium hydroxide, a potassium hydroxide or a calcium hydroxide or any combination thereof to raise the pH (make the liquid and/or semisolid or paste more alkaline) to:
(i) between about pH 9.5 and about pH 10.5,
(ii) a point at which substantially most of the protein flocculates or coagulates, or the protein and lipid form an emulsion, or
(iii) a point at which the protein and lipid form a lipid-encapsulated structure (a liposome or vesicle comprising the protein) or a protein-encapsulated structure,
and raising the temperature to: (i) at or above the melting point of milk fat, or to between about 120° F. to about 160° F., either before, during and/or after adding the base-comprising second liquid to the first liquid; and
(c) mixing and shearing the liquid of step (b), and at the same time or any time during the process of the mixing and shearing or sonicating, adding a liquid comprising an acid comprising a phosphoric acid to:
(i) lower the pH (make the liquid more acid) to: or between about pH to 3.0 to about 6.5, or between about pH 6.1 to about pH 6.3, or between about pH to 4.5 to about pH 3.3, thereby generating a precipitate or flocculant comprising substantially most of the protein, lipid or lipid-encapsulated structures or protein-encapsulated structures out of the liquid, or
(ii) lower the pH (make the liquid more acid) to the point where substantially most of the protein, lipid, or lipid-encapsulated structures or protein-encapsulated structures precipitate or flocculate out of the liquid;
(d) separating or removing the liquid from the precipitate or flocculate, or separating or removing the precipitate or flocculate from the liquid.

38-48. (canceled)

49. A method for processing and recovering organic matter from a dairy-based, soy-based and/or a fruit-based liquid, comprising:
(a) providing a dairy-based, soy-based and/or a fruit-based first liquid, colloid and/or semisolid or paste, wherein the first liquid, colloid and/or semisolid or paste comprises a protein;
(b) adding to the dairy-based, soy-based and/or a fruit-based first liquid, colloid and/or semisolid or paste of step (a) a second liquid comprising a base comprising a sodium hydroxide, a potassium hydroxide or a calcium hydroxide or any combination thereof to raise the pH (make the liquid and/or semisolid or paste more alkaline) to:
(i) between about pH 9.5 and about pH 10.5, or between about pH 10.5 and about pH 11.5, or
(ii) a point at which substantially most of the protein flocculates or coagulates;
and raising the temperature to: (i) at or above the melting point of milk fat, or to between about 120° F. to about 160° F., or between about 90° F. to about 130° F., either before, during and/or after adding the base-comprising second liquid to the first liquid;
(c) adding to and mixing into the raised temperature liquid of step (c) a preheated (prewarmed) liquefied fat or lipid, wherein the fat or lipid is preheated (prewarmed) to a temperature: (i) at or above the melting point of milk fat, or to between about 120° F. to about 160° F.;
(d) mixing and shearing the liquid of step (c), and at the same time or any time during the process of the mixing and shearing or sonicating, adding a liquid comprising an acid comprising a phosphoric acid to:
(i) lower the pH (make the liquid more acid) to: or between about pH to 3.0 to about 4.0, or between about pH 3.6 to about pH 4.2, or about pH 3.6, pH 3.7, pH 3.8, pH 3.9 or pH 4.0, thereby generating a precipitate or flocculant, or
(ii) lower the pH (make the liquid more acid) to the point where substantially most of the protein, fat or lipid, or fat/lipid-encapsulated structures or protein-encapsulated structures precipitate or flocculate out of the liquid; and
(e) separating or removing the liquid from the precipitate or flocculate, or separating or removing the precipitate or flocculate from the liquid.

50-79. (canceled)
